(19) United States
(12) Patent Application Publication
(10) Pub. No.: US 2009/0181390 A1
(43) Pub. Date: Jul. 16, 2009

(54) HIGH THROUGHPUT DETECTION OF MICRORNAS AND USE FOR DISEASE DIAGNOSIS

(75) Inventors: Xianqiang Li, Palo Alto, CA (US); Xin Jiang, Saratoga, CA (US)

Correspondence Address: WILSON SONSINI GOODRICH & ROSATI, 650 PAGE MILL ROAD, PALO ALTO, CA, 94304-1050, US

(73) Assignee: SIGNOSIS, INC. A CALIFORNIA CORPORATION

(21) Appl. No.: 12/263,152

(22) Filed: Oct. 31, 2008

Related U.S. Application Data
(60) Provisional application No. 61/020,693, filed on Jan. 11, 2008.

(51) Int. Cl. C12Q 1/68

(52) U.S. Cl. 435/6

(57) ABSTRACT

Methods, compositions and kits are provided for high throughput detection of micro RNAs (miRNA), especially for sensitive and specific detection of miRNA that are in low abundance and closely related to each other. In particular, an assembly of designed oligonucleotide probes with unique tag sequences is used to achieve these purposes via high throughput microarrays, optionally in conjunction with branched-DNA based array detection. The assays can be used for diagnosis, prognosis or monitoring of diseases or disorders such as cancer, for pharmacogenomic studies of patient stratification and drug responses, for discovery of therapeutic targets, or for forensic analysis.

### CROSS-REFERENCE

This application claims the benefit of U.S. Provisional Application No. 61/020,693, filed Jan. 11, 2008, which application is incorporated herein by reference in its entirety.

### BACKGROUND OF THE INVENTION

Nucleic acid analysis is becoming an important tool for the diagnosis and prognosis of infectious as well as genetic diseases. For instance, new discovered microRNAs (miRNAs) are important to the regulation of gene expression. These small molecules inhibit protein production through selective binding to the complementary messenger RNA sequences. Although the inhibition-mediated biological function of these miRNA molecules are not yet fully understood, miRNAs seems to be crucial in diverse regulations, including development, cell differentiation, proliferation, apoptosis, and maintenance of stemness and imprinting. Moreover, for an increasing number of genetic diseases, the genes involved have been identified and mutant alleles characterized.

Large-scale multiplex analysis of nucleic acid is needed for practical identification of individuals, e.g., for paternity testing and in forensic science, for organ-transplant donor-recipient matching, for genetic disease diagnosis, prognosis, and pre-natal counseling, and the study of oncogenic mutations. In addition, the cost-effectiveness of infectious disease diagnosis by nucleic acid analysis varies directly with the multiplex scale in panel testing. Many of these applications depend on the discrimination of single-base differences at a multiplicity of sometimes closely spaced loci.

Although there are many techniques currently used to detect target nucleic acids, the need remains for a rapid single assay format to detect the presence or absence of multiple selected sequences in a polynucleotide sample.

### SUMMARY OF THE INVENTION

The invention relates to methods, compositions and devices, e.g., for detecting a target nucleic acid in a sample.

In one aspect, the invention provides a method for detecting a target nucleic acid in a sample. In some embodiments of this aspect, the invention provides an oligonucleotide probe set. In some embodiments, the invention provides at least one oligonucleotide probe set, each set containing (i) a first oligonucleotide probe having a 5′ target specific region and a first 3′ universal sequence region, (ii) a second oligonucleotide probe having a 3′ target specific region and a second 5′ universal sequence region, (iii) a third oligonucleotide probe having a 5′ region complementary to the first 3′ universal sequence region in the first probe, and (v) a fourth oligonucleotide probe having a 3′ region complementary to the 5′ universal sequence region of the second oligonucleotide probe. In some embodiments, the first and the second oligonucleotides probes are suitable for ligation together when hybridized adjacent to one another to the target nucleic acid. In some embodiments, the third and the fourth oligonucleotides probes are suitable for ligation to the target nucleic acid when hybridized adjacent to the nucleic acid target. The oligonucleotide probe set is annealed to the target nucleic acid such that a complex is formed between the target nucleic acid and the oligonucleotide probe set, and the complex is contacted with a linking agent under conditions such that the directly adjacent 5′ and 3′ ends of the first and second oligonucleotide probes, the 3′ and 5′ ends of the third oligonucleotide probe and the target nucleic acid, and the 5′ and 3′ ends of the fourth oligonucleotide probe and the target nucleic acid covalently bond to form a ligated probe product. The ligated probe product is separated from the non-ligated first and second oligonucleotide probes, and the ligated probe product is detected, where the presence of the ligated product is indicative of presence of a target nucleic acid in the sample.

In some embodiments, the probes in the oligonucleotide probe set have a predetermined sequence. In some embodiments, the first oligonucleotide probe contains in a 3′ to 5′ order a universal region, a tag region and a target specific region. In some embodiments, the second oligonucleotide probe contains in a 3′ to 5′ order a target specific region, a tag region and a universal sequence region. In some embodiments, the third oligonucleotide probe contains a 3′ region that is complementary to the tag region of the first oligonucleotide probe. In some embodiments of the methods, the invention provides a fifth oligonucleotide probe that is complementary to the tag region of the first oligonucleotide probe. In some embodiments, the fourth oligonucleotide probe contains 5′ region that is complementary to the tag region of the second oligonucleotide probe. In some embodiments of the methods, the invention provides a sixth oligonucleotide probe that is complementary to the tag region of the second oligonucleotide probe.

In some embodiments, the tag region in the first oligonucleotide probe or the tag in the second oligonucleotide probe are specifically assigned to the target nucleic acid.

In some embodiments, at least one of the universal regions of the first and the second oligonucleotide probe is a promoter sequence. The promoter sequence can be used as a primer of DNA polymerase. Examples of DNA polymerase include, but are not limited to, Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase. In some embodiments, the promoter sequence is a promoter for a phage polymerase. Examples of phage polymerase include, but are not limited to, T7 RNA polymerase, T3 RNA polymerase or SP6 RNA polymerase.

In some embodiments of the methods, the invention includes annealing a first primer complementary to the universal sequence region of the first oligonucleotide probe, and contacting the annealed primer with a polymerase under conditions such that the annealed primer is extended to form an extension product complementary to the sequences to which the primers is annealed. In some embodiments, the presence of the extension product is detected, where the presence of the extended product is indicative of the presence of the target nucleic acid in the sample. In some embodiments of the methods, the invention includes annealing a second primer complementary to the universal sequence region of the fourth oligonucleotide probe, and contacting the annealed second primer with a polymerase under conditions such that the annealed primer is extended to form an extension product complementary to the sequences to which the primers is annealed. In some embodiments, the presence of the extension product is detected, where the presence of the extended product is indicative of the presence of the target nucleic acid in the sample.

In some embodiments of the methods, the invention includes annealing a first primer complementary to the universal sequence region of the fourth oligonucleotide probe, contacting the annealed primer with a polymerase under conditions such that the annealed primer is extended to form extension products complementary to the sequences to which the primers is annealed. In some embodiments, the presence of the extension product is detected, where the presence of the extended product is indicative of the presence of the target nucleic acid in the sample.

The extension products can be detected using a DNA microarray, bead microarray, high throughput sequencing or single microtiter plate assay. In some embodiments, the extension product has a detectable label. The detectable label can be a fluorescent or biotin label. In some embodiments, the invention includes detecting a fluorescent signal generated by the fluorescent, chemiluminescent or color. In some embodiments, the label is attached to the primer complementary to the universal sequence region of the first oligonucleotide probe. In some embodiments, the label is incorporated during the extension of the annealed primer complementary to the universal sequence region of the first oligonucleotide probe. In some embodiments, the incorporation includes adding a label nucleotide to the extension of the annealed primer complementary to the universal sequence region of the third oligonucleotide probe.

In some embodiments, the universal sequence region of the second oligonucleotide is a phage promoter. Examples of phage promoters include, but are not limited to, T7 RNA polymerase promoter, T3 RNA polymerase promoter or SP6 RNA polymerase promoter. In some embodiments, the phage promoter is a T7 RNA polymerase promoter.

In some embodiments of the methods, the invention includes contacting the phage promoter region of the second oligonucleotide probe with a phage polymerase under conditions such that a transcription product of the phage promoter region is formed and detecting the presence of the transcription product, where the presence of the transcription product is indicative of the presence of the target nucleic acid in the sample. In some embodiments, the transcription product is detected using a DNA microarray, bead microarray, high throughput sequencing or a single microtiter plate assay.

In some embodiments, the transcription product has a detectable label. The detectable label can be a fluorescent or biotin label. In some embodiments, the invention includes detecting a fluorescent signal generated by the fluorescent or chemiluminescent or color. In some embodiments the label is incorporated during the transcription of the phage promoter region of the second oligonucleotide probe. In some embodiments, the incorporation includes adding a label nucleotide to the transcription of the phage promoter region of the second oligonucleotide probe.

In some embodiments, the target nucleic acid is a miRNA molecule. In some embodiments, the miRNA molecule is derived from total RNA.

In some embodiments, the first or third oligonucleotide contains a capturing portion. The capturing portion can be used to separate the ligated probe product from unligated first and second oligonucleotide probes. Examples of capturing portions include, but are not limited to, biotin and a capture sequence. In some embodiments, the capturing portion is biotin. In some embodiments, the ligated probe product is isolated by binding the biotin with a strepavidin bound to a solid support.

In some embodiments of the methods, the invention provides a loop that links the second and the fourth oligonucleotide. In some embodiments, the invention includes detecting the presence of a ligated probe containing a loop to indicate the presence of the target nucleic acid in said sample. In some embodiments, detecting the presence of a ligated probe containing a loop includes binding a branched DNA to the ligated probe. In some embodiments, the ligated probe containing a loop is detected using a DNA microarray, bead microarray, or high throughput sequencing.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates an embodiment of the invention of miRNA annealing with two stacking oligos to form a miRNA/DNA hybrid

FIG. 2: schematically illustrates an embodiment of the invention of miRNA forming a complex with four oligos stacked together

FIG. 3: schematically illustrates an embodiment of the invention for probe preparation of miRNA

FIG. 4 schematically illustrates an embodiment of the invention for a hair pin after ligation of miRNA complex with four oligos stacked together.

FIG. 5 schematically illustrates an embodiment of the invention for analysis of miRNA.

FIG. 6 schematically illustrates an embodiment of the invention for bDNA detection in a miRNA array assay

FIG. 7 schematically illustrates an embodiment of the invention for probe preparation for bDNA detection using a complex with four oligos stacked together with the target nucleic acid

FIG. 8 schematically illustrates an embodiment of the invention for analysis of miRNA.

FIG. 9 shows the sequences for Let-7a, Let-7b and Let-7c miRNA in A, and shows discrimination of let 7 miRNA with a microarray in B.

FIG. 10 shows an array profiling miRNA expression in HeLa cells with T7 transcription and PCR amplification.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entirety.

The assay of the present invention is particularly useful for analyzing nucleic acids (DNA or RNA). The methods described herein provide a sensitive assay for determining the presence or absence of a target nucleic acid, e.g., the presence of absence of a point mutation or a SNP in a target nucleic acid. In some embodiments, the method described herein use oligonucleotide probes which are complementary to two contiguous predetermined sequences of the test substance. If these probes anneal in a juxtaposed position, there is a reasonable certainty that the sequence being investigated is the relevant one. The annealed probes are then exposed to a linking agent which then ligates the adjacent ends of the probes if the nucleotides base pair at the target nucleotide position. Then, the presence or absence of ligation is determined by one of a number of techniques to be described below.

The oligonucleotide probe sets can be in the form of any nucleotide such as ribonucleotides, deoxynucleotides, modified ribonucleotides, modified deoxyribonucleotides, peptide nucleotide analogues, modified peptide nucleotide analogues, modified phosphate-sugar-backbone oligonucleotides, nucleotide analogs, and mixtures thereof. In some embodiments, the oligonucleotide probe sets are in the form of deoxynucleotides.

The linking agent could be a ligase. In some embodiments the ligase is T4 DNA ligase, using well known procedures (Maniatis, T. in Molecular Cloning, Cold Spring Harbor Laboratory (1982)). Other DNA ligases may also be used. T4 DNA ligase may also be used when the target nucleic acid is RNA (The Enzymes, Vol. 15 (1982) by Engler M. J. and Richardson C. C., p. 16-17. Methods in Enzymology, Vol. 68 (1979) Higgins N. P. and Cozzarelli N. R. p. 54-56). With regard to ligation, other ligases, such as those derived from thermophilic organisms may be used thus permitting ligation at higher temperatures allowing the use of longer probes (with increased specificity) which could be annealed and ligated simultaneously under the higher temperatures normally associated with annealing such probes. The ligation, however, need not be by an enzyme and, accordingly, the linking agent may be a chemical agent which will cause the probes to link unless there is a nucleotide base pair mismatching at the target nucleotide position. For simplicity, some embodiments of the invention will be described using T4 DNA ligase as the linking agent. This enzyme requires the presence of a phosphate group on the 5′ end that is to be joined to a 3′ OH group on a neighboring oligonucleotide.

In some cases, the methods described herein involve performing one or more genetic analyses or detection steps on nucleic acids. In some embodiments target nucleic acids are from a sample obtained from an animal. Such animal can be a human or a domesticated animal such as a cow, chicken, pig, horse, rabbit, dog, cat, or goat. Samples derived from an animal, e.g., human, can include, for example whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. In some embodiments the sample is a cell sample. Cell samples can be obtained from a variety of tissues depending on the age and condition of the animal. Cell samples can be obtained from peripheral blood using well known techniques. In fetal testing, a sample can be obtained by amniocentesis, chorionic villi sampling or by isolating fetal cells from the blood of a pregnant individual. Other sources of nucleic acids include blood, semen, buccal cells, or the like. Nucleic acids can be obtained from any tissue or organ by methods well known in the art.

To obtain a blood sample, any technique known in the art may be used, e.g. a syringe or other vacuum suction device. A blood sample can be optionally pre-treated or processed prior to enrichment. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent.

When a blood sample is obtained, a preservative such an anti-coagulation agent and/or a stabilizer can be added to the sample prior to enrichment. This allows for extended time for analysis/detection. Thus, a sample, such as a blood sample, can be analyzed under any of the methods and systems herein within 1 week, 6 days, 5 days, 4 days, 3 days, 2 days, 1 day, 12 hrs, 6 hrs, 3 hrs, 2 hrs, or 1 hr from the time the sample is obtained.

In some embodiments, a blood sample can be combined with an agent that selectively lyses one or more cells or components in a blood sample. For example, fetal cells can be selectively lysed releasing their nuclei when a blood sample including fetal cells is combined with deionized water. Such selective lysis allows for the subsequent enrichment of fetal nuclei using, e.g., size or affinity based separation. In another example platelets and/or enucleated red blood cells are selectively lysed to generate a sample enriched in nucleated cells, such as fetal nucleated red blood cells (fnRBC) and maternal nucleated blood cells (mnBC). The fnRBC's can subsequently be separated from the mnBC's using, e.g., affinity to antigen-i or magnetism differences in fetal and adult hemoglobin.

When obtaining a sample from an animal (e.g., blood sample), the amount can vary depending upon animal size, its gestation period, and the condition being screened. In some embodiments, up to 50, 40, 30, 20, 10, 9, 8, 7, 6, 5, 4, 3, 2, or 1 mL of a sample is obtained. In some embodiments, 1-50, 2-40, 3-30, or 4-20 mL of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

Nucleic acids from samples that can be analyzed by the methods herein include: double-stranded DNA, single-stranded DNA, single-stranded DNA hairpins, DNA/RNA hybrids, RNA (e.g. mRNA or miRNA) and RNA hairpins. Examples of genetic analyses that can be performed on nucleic acids include e-g., SNP detection, STR detection, RNA expression analysis, promoter methylation, gene expression, virus detection, viral subtyping and drug resistance.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

In some embodiments, the methods described herein are used to detect and/or quantified a target nucleic acid molecule. In some embodiments, the methods described herein are used to detect and/or quantified multiple target nucleic acid molecules. The methods described herein can analyzed at least 1, 2, 3, 4, 5, 10, 20, 50, 100, 200, 500, 1,000, 2,000, 5,000, 10,000, 20,000, 50,000, 100,000, different target nucleic acids.

In some embodiments, the methods described herein are used to detect and/or quantify target nucleic acids to profile a specific tissue or a specific condition. In some embodiments, the methods described herein are used to detect and/or quantify target nucleic acids to detect biomarkers for specific tissue or condition. In some embodiments, the methods described herein are used to regulate gene expression. In some embodiments, the methods described herein are use for gene therapy. In some embodiments, the methods described herein are used to detect and/or quantify target nucleic acids to profile a neoplastic and/or cancer cell. In some embodiments, the methods described herein are used to detect and/or quantify target nucleic acids to diagnose cancer and/or a neoplastic condition. In some embodiments, the methods described herein are used to detect and/or quantify target nucleic acids to detect biomarkers in a neoplastic and/or cancer cell. In some embodiments, the methods described herein are used to regulate gene expression in a neoplastic and/or cancer cell. In some embodiments, the methods described herein are used for gene expression.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

In some embodiments, the methods described herein are used to quantify nucleic acid expression in different tissues, developmental lineages and/or different states of a condition. In some embodiments, the methods described herein are used to quantify nucleic acid expression in different states of a neoplastic and/or cancer condition.

In some embodiments, the methods described herein are used to detect and/or quantify target nucleic acids without the need of target nucleic acid isolation. In some embodiments, the methods described herein are used to detect and/or quantify a target nucleic acid directly from a nucleic acid sample comprising DNA and RNA molecules.

In some embodiments, the methods described herein are used to quantify nucleic acid expression in different tissues, developmental lineages and/or different states of a condition. In some embodiments, the methods described herein are used to quantify nucleic acid expression in different states of a neoplastic and/or cancer condition.

In some embodiments, the methods described herein are used to distinguish between target nucleic acids that differ from another nucleic acid by 1 nt. In some embodiments, the methods described herein are used to distinguish between target nucleic acids that differ from another nucleic acid by 1 nt or more than 1, 2, 3, 5, 10, 15, 20, 21, 22, 24, 25, 30 nt.

In some embodiments, the methods described herein are used to detect and/or quantified multiple target nucleic acid molecules. The methods described herein can analyzed at least 1, 2, 3, 4, 5, 10, 20, 50, 100, 200, 500, 1,000, 2,000, 5,000, 10,000, 20,000, 50,000, 100,000, different target nucleic acids.

In some embodiments, the methods described herein are used to detect and/or quantify target nucleic acids without the need of target nucleic acid isolation. In some embodiments, the methods described herein are used to detect and/or quantify a target nucleic acid directly from a nucleic acid sample comprising DNA and RNA molecules.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

miRNA

In some embodiments, the methods described herein are used to detect and/or quantify microRNAs (miRNAs). New discovered miRNAs are thought to be important in the regulation of gene expression. MiRNA are usually single-stranded RNAs approximately 22 nt long. Without being limited to any theory, these small molecules inhibit protein production through selective binding to the complementary messenger RNA sequences. Although the inhibition-mediated biological function of these miRNA molecules are not yet fully understood, miRNAs seems to be crucial in diverse regulations, including development, cell differentiation, proliferation, apoptosis, and maintenance of stemness and imprinting. Through selective binding to complementary messenger RNA sequences, they can mediate translation repression or RNA degradation. Up to 20%-25% of mammalian genes might be regulated by miRNAs. So far, more than 400 miRNAs have been identified in human genome. Many of them are only different in one or few nucleotides (http://microrna.sanger.ac.uk/sequences/ftp.shtml).

In some embodiments, the methods described herein are used to detect and/or quantified a miRNA molecule. In some embodiments, the methods described herein are used to detect and/or quantified multiple miRNA molecules. The methods described herein can analyzed at least 1, 2, 3, 4, 5, 10, 20, 50, 100, 200, 500, 1,000, 2,000, 5,000, 10,000, 20,000, 50,000, 100,000, different miRNAs.

Recent studies have shown that expression of mature miRNAs is tissue-specific and the abundance of miRNAs varies several orders of magnitude. In some cases, expression of miRNAs is tissue-specific, such as the expression of miRNAs miR-1 and miR-133 to be specific to heart and skeletal muscle and miR-122a specific to liver tissue. Moreover, miss-regulation of miRNA expression might contribute to human cancers and miRNAs are considered to be a new class of cellular molecules involved in human oncogenesis. miRNA has been demonstrated to be a new class of cellular molecules involved in human oncogenesis. The first report was made in chronic lymphatic leukemia (CLL) where a number of patients show down-regulation of miRNA-15 and miRNA-16. These studies were followed by studies demonstrating altered expression of miRNA in a number of cancers including colon cancers, Burkitt lymphoma, lung cancer, breast cancer, large cell lymphoma, glioblastoma, B cell lymphoma, hepatocellular carcinoma, and papillary thyroid carcinoma. Expression of mature miRNA is also found to be specific to normal but not cancer cells and tissues. For instance, the expression of mature miR-122a is very low in four liver cancer cell lines and hepatocellular carcinomas, but very high in normal liver tissue. Systemically profiling of miRNA expression displays unique signatures in a number of cancers, such as the difference that can differentiate malignant and non-malignant prostate samples, and discriminate clinically relevant breast cancer phenotypes.

In some embodiments, the methods described herein are used to detect and/or quantify miRNAs to profile a specific tissue or a specific condition. In some embodiments, the methods described herein are used to detect and/or quantify miRNAs to detect biomarkers for specific tissue or condition. In some embodiments, the methods described herein are used to regulate gene expression. In some embodiments, the methods described herein are use for gene therapy. In some embodiments, the methods described herein are used to detect and/or quantify miRNAs to profile a neoplastic and/or cancer cell. In some embodiments, the methods described herein are used to detect and/or quantify miRNAs to diagnose cancer and/or a neoplastic condition. In some embodiments, the methods described herein are used to detect and/or quantify miRNAs to detect biomarkers in a neoplastic and/or cancer cell. In some embodiments, the methods described herein are used to regulate gene expression in a neoplastic and/or cancer cell. In some embodiments, the methods described herein are used for gene expression.

miRNAs are found in the genomes of humans, animals, plants and viruses. miRNAs are generated from endogenous hairpin-shaped transcripts. In animals, miRNAs are transcribed as long primary transcripts (pri-microRNAs) by RNA polymerase II enzyme. They are cleaved in the nucleus by RNAse III endonuclease Drosha, releasing a ˜60-70 nt stem loop pre-miRNAs. The pre-miRNA is actively transported to the cytoplasm by export receptor exportin-5 where it is processed by the enzyme Dicer yielding a 22 nt microRNA duplexes. Following denaturation by the action of helicases, one strand of the duplex (the mature miRNA) is incorporated into a ribonucleoprotein complex known as RISC(RNA-induced silencing complex), which will guide the particular miRNA to its messenger RNA target to lead to regulation of the corresponding protein. In some embodiments, the methods described herein are used to distinguish precursors miRNA from mature miRNA. The methods described herein can distinguish at least 1, 2, 3, 4, 5, 10, 20, 50, 100, 200, 500, 1,000, 2,000, 5,000, 10,000, 20,000, 50,000, 100,000, different miRNAs from their precursor.

Many miRNA have been identified through both biological approach and informatics analysis. There are total 475 human miRNA genes listed in the miRNA database (http://microrna.sanger.ac.uk/sequences/ftp.shtml) and it is expected to be approximately 1000, which would be equivalent to almost 3% of the protein-coding genes. Many of mature human miRNAs are closely related in sequences and more than 20% are grouped into isoforms with nearly identical sequences, usually differing by 1-3 nt. The largest human isoform families include let-7, including 9 mature molecules with different sequences. These families are designated with a letter (e.g. let-7b and let-7c). Because of the minor difference of isoforms in addition to the small size of the molecules and coexistence with precursors, it is quite challenge to analyze or profile miRNAs. In some embodiments, the methods described herein are used to distinguish between miRNA isoforms. In some embodiments, the methods described herein are used to distinguish between miRNA isoforms that differ by 1 nt. In some embodiments, the methods described herein are used to distinguish between miRNA isoforms that differ by more than 1, 2, 3, 5 nt. The methods described herein can distinguish between at least 1, 2, 3, 4, 5, 10, 20, 50, 100, 200, 500, 1,000, 2,000, 5,000, 10,000, 20,000, 50,000, 100,000, different miRNAs isoforms.

Distinguished miRNA expression has found in the different tissues, developmental lineages and differentiation states of various human malignancies. In some embodiments, the methods described herein are used to quantify miRNA expression in different tissues, developmental lineages and/or different states of a condition. In some embodiments, the methods described herein are used to quantify miRNA expression in different states of a neoplastic and/or cancer condition.

In some embodiments, the method described herein are used to detect and/or quantify miRNA when less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample. In some cases, about 1-5 pg, 5-1 0 pg, or 10-100 pg of nucleic acids are obtained from the sample for further analysis according to methods described herein.

In some embodiments, the methods described herein are used to detect and/or quantify miRNA without the need of miRNA isolation. In some embodiments, the methods described herein are used to detect and/or quantify miRNA without the need of RNA isolation. In some embodiments, the methods described herein are used to detect and/or quantify miRNA directly from a nucleic acid sample comprising DNA and RNA molecules.

### Oligonucleotide Ligation Assay

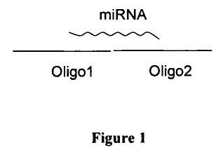

In one aspect of the invention a set of oligonucleotides is designed to detect a target nucleic acid. FIG. 1 shows an embodiment of the invention in which a pair of oligonucleotides (depicted as oligo 1 and oligo 2 in FIG. 1) binds to a target miRNA. Even though FIG. 1 depicts a process for miRNA detection, the methods described herein can also be used in other analysis, including STR and SNP detection, RNA expression analysis, promoter methylation, gene expression, virus detection, viral subtyping and drug resistance.

In some embodiments the set of oligonucleotide probes comprises a first oligonucleotide probe having a 5′ target specific region and a 3′ universal sequence region (depicted as oligo 1 in FIG. 1), and a second probe having a 3′ target specific region and a 5′ universal promoter region (depicted as oligo 2 in FIG. 1). In some embodiments, the first and the second oligonucleotide probes are suitable for ligation together when hybridized adjacent to one another to said target nucleic acid as shown in FIG. 1.

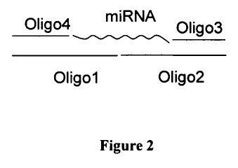

In some embodiments the set of oligonucleotide probes comprises a first oligonucleotide probe having a 5′ target specific region and a 3′ universal sequence region (depicted as oligo 1 in FIG. 2), a second probe having a 3′ target specific region and a 5′ universal sequence region (depicted as oligo 2 in FIG. 2), a third oligonucleotide probe having a 5′ region specific to the first 3′ universal sequence region in the first probe (depicted as oligo 4 in FIG. 2), and a fourth probe having a 3′ region specific to the 5′ universal sequence region of said second oligonucleotide probe (depicted as oligo 3 in FIG. 2). In some embodiments, the first and said second oligonucleotides probes are suitable for ligation together when hybridized adjacent to one another to said target nucleic acid, and the third and the fourth oligonucleotides probes are suitable for ligation to the target nucleic acid when hybridized adjacent to said nucleic acid target. Specifically, the 5′ region of the first oligonucleotide probe anneals to the 5′ region of said target nucleic acid such that the target specific region of said first oligonucleotide probe is aligned with the 5′ region of said target nucleic acid and the 3′ region of the second oligonucleotide probe anneals to the 3′ region of said target nucleic acid such that the target specific region of said second oligonucleotide probe is aligned with the 3′ region of said target nucleic acid. Furthermore, the 3′ end of the third oligonucleotide probe and the 5′ end of the fourth oligonucleotides probe are suitable for ligation to target nucleic acid when the 3′ end of the third oligonucleotide is hybridized adjacent to the 5′ end of the target nucleic acid and the 5′ end of the fourth oligonucleotides probe is hybridized adjacent to the 3′ downstream end of the target nucleic acid.

In some embodiments, the set of oligonucleotide probes comprises a first oligonucleotide probe having a 5′ target specific region and a 3′ universal sequence region (depicted as oligo 1 in FIG. 1 and FIG. 2). The universal region in oligo 1 can be the sequence of a promoter. In some embodiment, the promoter sequence in oligo 1 is a promoter for a DNA polymerase. Examples of DNA polymerase include, but are not limited to, Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4DNA polymerase, bacteriophage PR5DNA polymerase, bacteriophage PR722DNA polymerase and bacteriophage L17 DNA polymerase. In some embodiments, the promoter sequence in oligo 1 is a promoter for a phage polymerase. Examples of phage polymerase include, but are not limited to, T7 RNA polymerase, T3 RNA polymerase or SP6 RNA polymerase. In some embodiments, the universal sequence can be used to capture or detect the oligonucleotide set as described herein.

In some embodiments, the set of oligonucleotide probes comprises a second oligonucleotide probe having a 3′ target specific region and a 5′ universal region (depicted as oligo 2 in FIG. 1 and FIG. 2). The universal region in oligo 1 can be the sequence of a promoter as described above. In some embodiments, the universal sequence can be used to capture or detect the oligonucleotide set as described herein.

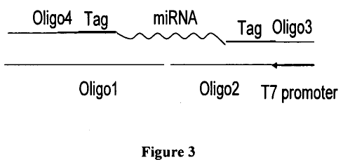

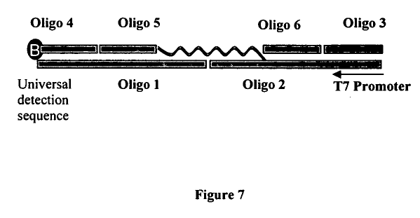

In some embodiments, the set of oligonucleotide probes comprises a first oligonucleotide probe having in a 3′ to 5′ order a universal sequence region, a tag region and a target specific region (depicted as oligo 1 in FIGS. 1 and 2), where the universal region can be a promoter as described above. In some embodiments, the universal sequence can be used to capture or detect the oligonucleotide set as described herein. In some embodiments, the tag region of oligo 1 can be a unique sequence assigned to a specific target nucleic acid. The tag sequence can be used to capture or detect the oligonucleotide set as described herein. In some embodiments where the first oligonucleotide has a tag region, the third oligonucleotide probe has a 3′ region that is specific to the tag region of the first oligonucleotide probe (depicted as oligo 4 in FIG. 3). In some embodiments where the first oligonucleotide has a tag region, the probe set further comprises a fifth oligonucleotide probe that is specific to the tag region of said first oligonucleotide probe (depicted as oligo 5 in FIG. 7).

In some embodiments, the set of oligonucleotide probes comprises a second oligonucleotide probe having in a 5′ to 3′ order a universal sequence region, a tag region and a target specific region (depicted as oligo 2 in FIGS. 1 and 2), wherein the universal sequence region can be a promoter region as described above. In some embodiments, the tag region of oligo 2 can be a unique sequence assigned to a specific target nucleic acid. The tag sequence can be used to capture or detect the oligonucleotide set as described herein. In some embodiments where the second oligonucleotide has a tag region, the fourth oligonucleotide probe has a 5′ region that is specific to the tag region of the second oligonucleotide probe (depicted as oligo 3 in FIG. 3). In some embodiments where the second oligonucleotide has a tag region, the probe set further comprises a sixth oligonucleotide probe that is specific to the tag region of said second oligonucleotide probe (depicted as oligo 6 in FIG. 7).

For simplicity, most of the examples and embodiments of the invention will be illustrated using a set of oligonucleotide probes containing a first, a second, a third and a fourth probe named oligo 1, oligo 2, oligo 4 and oligo 3 throughout the examples and embodiments described herein. However, as described above a probe set containing less than four probes or more than four probes are encompassed in the methods described herein. In some embodiments, at least three oligonucleotides are designed to detect a target nucleic acid. In some embodiments, two pairs of oligonucleotides are designed to detect a target nucleic acid. In some embodiments, three pairs of oligonucleotides are designed to detect a target nucleic acid.

In some embodiments, a set of two oligonucleotide probes binds to a target nucleic acid (as depicted in FIG. 1 as oligo 1 and 2). In some embodiments, a set of four oligonucleotide probes binds to a target nucleic acid (as depicted in FIG. 2 as oligos 1-4). In some embodiments, any of the oligos have a capturing portion to separate the oligos bound to the target nucleic acid. The capturing portion can be a marker or a capturing sequence.

In some embodiments, the capturing portion is a capturing sequencing. The capturing sequence can be the universal sequence of oligo 1 or oligo 2 or the tag sequence of either oligo 1 or 2 as described above. The capturing sequence can be a new portion in oligo 1 or oligo 2 distinct from the universal sequence or tag sequences described above. The capturing sequence can be the universal sequence of oligo 3 or oligo 4 or the tag sequence of either oligo 3 or 4 as described above. The capturing sequence can be a new portion in oligo 3 or oligo 4 distinct from the universal sequence or tag sequences described above. In some embodiments, a capturing sequence is introduced at oligo 1, which can be captured by capturing sequence-conjugated to a solid structure such as beads or an oligonucleotide array. In some embodiments, a capturing sequence is introduced at oligo 2, which can be captured by capturing sequence-conjugated to a solid structure such as beads or an oligonucleotide array. In some embodiments, a capturing sequence is introduced at oligo 3, which can be captured by capturing sequence-conjugated to a solid structure such as beads or an oligonucleotide array. In some embodiments, a capturing sequence is introduced at oligo 4, which can be captured by capturing sequence-conjugated to a solid structure such as beads or an oligonucleotide array.

In some embodiments, the capturing portion is a marker. Markers that are used to capture oligos are known in the art. The marker then can be captured by in a subsequent isolation step by a marker-binding solid structure. In some embodiments the marker is biotin. In some embodiments, biotin is introduced at oligo 1, which can be captured by streptavidin-conjugated to a solid structure such as beads. In some embodiments, biotin is introduced at oligo 2, which can be captured by streptavidin-conjugated to a solid structure such as beads. In some embodiments, biotin is introduced at oligo 3, which can be captured by streptavidin-conjugated to a solid structure such as beads. In some embodiments, biotin is introduced at oligo 4, which can be captured by streptavidin-conjugated to a solid structure such as beads. Biotin can be introduced at any of the oligos by annealing a primer containing biotin to the universal sequence of the oligos. Alternatively, biotin can be introduced at any of the oligos when the oligos are synthesized by methods known in the art.

In some embodiments, oligo 1 will have a phosphate group at its 5′ end. In some embodiments, oligo 1 and oligo 3 will have a phosphate group at its 5′ end. Optionally, oligo 2 will have a T7 promoter at its 5′ end. In some embodiments, when oligo 1 and oligo 2 simultaneously bind to one target nucleic acid molecule, e.g., miRNA, they are ligated according to techniques to known in the art. In some embodiments, when oligo 1 and oligo 2 simultaneously bind to one target nucleic acid molecule, and oligo 4 and oligo 3 bind to oligo 1 and 2, respectively, they are ligated according to techniques to known in the art. For example, the oligos can be ligated by T4 DNA ligase. When oligos are stacking together to bind to a molecule with a perfect match at the junction at their ends, it results in a specific binding to the targeted nucleic acid, e.g., mRNA. The stacking oligos can be ligated to form a ligated product, which can be used for detection. Without being limited to any theory, any sequence-closely related to the target nucleic acid molecules will either block the ligation or prevent the hybrid formation. Therefore, isoforms can be distinguished in the assay. If the difference is in the middle of the target nucleic acid, it will block the ligation and detection, although the hybrids are able to form. In some embodiments multiple nucleic acids are analyzed by mixing multiple oligo sets together, each of which is specific to one nucleic acid target.

In some embodiments, either one of oligo 1, oligo 2, oligo 3 or oligo 4 have a capturing portion to separate the oligos as described above. In some embodiments, after oligo 1 and 2, or oligo 1, oligo 2, oligo 3 and oligo 4 have been ligated the ligated product will be separated using the capturing portion in any of the oligos. After separation, the ligated products can then detached from the duplexes and analyzed according to the methods described herein

In some embodiments, the use of the set of oligonucleotides probes described herein allow for the detection of mature nucleic acids. For instance, miRNA precursors contain the identical sequence of a mature miRNA, hence, they can be targeted by the pair oligos represented as oligo 1 and 2 in FIG. 1, leading to ligation and detection. Either oligo 1 or 2 can be extended with a universal sequence and/or a unique tag sequence, corresponding to the sequence of oligo 3 or oligo 4. The oligo with the extended universal sequence and/or unique tag sequence will form a partial duplex with a protruding sequence, which is able to hybrid a part of a target nucleic acid molecule. If the target is a mature nucleic acid, e.g., miRNA molecule, the hybridization forms a perfect match with the oligo 3 or oligo 4 at the end of the miRNA, leading to a ligation. If the target is a precursor miRNA, no perfect match end is formed at the junction between the precursor and oligo 3 or oligo 4, and therefore no ligation can occur and no detection can be made. Furthermore, any nucleotide difference that exists at the ends among miRNA isoforms will result in imperfect match, which blocks either the formation of a hybrid or the ligation. In some embodiments, either oligo 1 and 2 can be extended with a unique tag sequence, corresponding to a region of oligo 4 and oligo 3, respectively (see FIG. 3). In some embodiments, either oligo 1 and 2 can be extended with a unique tag sequence, corresponding to the sequences of oligo 5 and oligo 6, respectively (see FIG. 7)

In some embodiments, at least two pairs of oligonucleotides allow for the detection of mature nucleic acids. For instance, miRNA precursors can be targeted by the pair oligos represented as oligo 1 and 2 in FIGS. 1 and 2, leading to ligation and detection. Oligo 1 and 2 are extended with universal sequences and/or two unique tag sequences, corresponding to the sequence of oligo 3 and 4 (FIG. 2). These oligos form two partial duplexes with a protruding sequence, each of which is able to hybrid a part of a target nucleic acid molecule. If the target is a mature nucleic acid, e.g, miRNA molecule, the hybridization forms two perfect matches with oligo 3 and oligo 4 at the ends of the miRNA (FIG. 2), leading to a ligation. If the target is a precursor miRNA, no perfect match ends are forming at the junctions between the precursor and oligo 3 and/or between the precursor and oligo 4, and therefore no ligation can occur and no detection can be made. Furthermore, any nucleotide difference that exists at the ends among miRNA isoforms will result in imperfect match, which blocks either the formation of a hybrid or the ligation.

In some embodiments, at least three pairs of oligonucleotides allow for the detection of mature nucleic acids. For instance, miRNA precursors can be targeted by the pair oligos represented as oligo 1 and 2 in FIGS. 1 and 2, leading to ligation and detection. Oligo 1 and 2 are extended with universal sequences and two unique tag sequences, corresponding to the sequence of oligo 3, 4, 5 and 6 (FIG. 7). These oligos form two partial duplexes with a protruding sequence, each of which is able to hybrid a part of a target nucleic acid molecule. If the target is a mature nucleic acid, e.g, miRNA molecule, the hybridization forms two perfect matches with oligo 5 and oligo 6 at the ends of the miRNA (FIG. 7), leading to a ligation. If the target is a precursor miRNA, no perfect match ends are forming at the junctions between the precursor and oligo 5 and/or between the precursor and oligo 6, and therefore no ligation can occur and no detection can be made. Furthermore, any nucleotide difference that exists at the ends among miRNA isoforms will result in imperfect match, which blocks either the formation of a hybrid or the ligation.

In some embodiments multiple nucleic acids are analyzed by mixing multiple oligo sets together, each of which is specific to one nucleic acid target. Each nucleic acid molecule will initiate the formation of a duplex and multiple nucleic acid lead to the assembly of multiple duplexes.

For simplicity, some of the embodiments described herein used two pairs of oligonucleotides to detect the target nucleic acids. However, the invention encompasses the use of at least 3, 4, 5, 6, 7, 8 9, 10 oligonucleotides to detect a target nucleic acid.

Many of the applications of the genetic analysis described herein depend on the discrimination of single-base differences between target nucleic acids. In some embodiments, to distinguish between nucleic acid targets that differ by one single nucleotide two pairs of oligos are used for the detection of target nucleic acid (depicted as oligo 1-4 in FIG. 2). A unique tag sequence is assigned in oligo 3 and/or oligo 4 for each specific target nucleic acid, depending on the location of the different nucleotide. That is the target nucleic acid can be targeted by the pair oligos represented as oligo 1 and 2 in FIGS. 1 and 2. Oligo 1 and 2 are then extended with two unique tag sequences, corresponding to the sequence of oligo 3 and 4. These tag sequences are assigned to a specific target nucleic acid. These tag sequences becomes new markers for the target nucleic acids, which can be easily differentiated by the method described herein. The oligos can then be ligated and analyzed as described herein.

In some embodiments, to distinguish between nucleic acid targets that differ by one single nucleotide six oligos are used for the detection of target nucleic acid as depicted in FIG. 7 (depicted as oligo 1-6 in FIG. 7). A unique tag sequence is assigned in oligo 5 and/or oligo 6 for each specific target nucleic acid, depending on the location of the different nucleotide. That is the target nucleic acid can be targeted by the pair oligos represented as oligo 1 and 2 in FIG. 7. Oligo 1 and 2 are then extended with two unique tag sequences (corresponding to the sequence of oligo 5 and 6) and two universal sequences (corresponding to the sequence of oligo 3 and 4). These tag sequences are assigned to a specific target nucleic acid. These tag sequences becomes new markers for the target nucleic acids, which can be easily differentiated by the method described herein. The universal sequences can be used for amplification, detection and/or capturing of the oligos as described in the methods herein. The oligos can then be ligated and analyzed as described herein.

In some embodiments, oligos/target nucleic acid hybrids are separated from free oligos and unhybridized nucleic acids prior to their analysis by the methods described herein. Two pairs of oligos can be used for the detection of target nucleic acid (FIG. 2). In some embodiments, six oligos are used (FIG. 7). In some embodiments, in order to separate the hybrids from the free oligos a capturing portion is introduced in at least one of the oligos, which can be captured by in a subsequent isolation step by a capturing portion-binding solid structure. The capture can be a marker or a predetermined capturing sequence as described above. In some embodiments, biotin is introduced at oligo 4 (FIG. 2 or FIG. 7), which can be captured by streptavidin-conjugated to a solid structure such as beads. In some embodiments, a capturing sequence is introduced at oligo 4 (FIG. 2 or FIG. 7), which can be captured by capturing sequence-conjugated to a solid structure such as beads or an oligonucleotide array. After separation, the ligated products can then detached from the duplexes and analyzed according to the methods described herein.

In some embodiments, the ligated products are amplified and optionally results are compared with amplification of similar target nucleic acids from a reference sample. In some embodiments, the ligated products of oligo 1 and oligo 2 are amplified and optionally results are compared with amplification of similar target nucleic acids from a reference sample. Amplification can be performed by any means known in the art. In some cases, the ligated products are amplified by polymerase chain reaction (PCR). Examples of PCR techniques that can be used include, but are not limited to, quantitative PCR, quantitative fluorescent PCR (QF-PCR), multiplex fluorescent PCR (MF-PCR), real time PCR (RTPCR), single cell PCR, restriction fragment length polymorphism PCR (PCR-RFLP), PCK-RFLPIRT-PCR-IRFLP, hot start PCR, nested PCR, in situ polonony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In any of the embodiments, amplification of ligated products may occur on a bead. In any of the embodiments herein, target nucleic acids may be obtained from a single cell.

In any of the embodiments herein, the nucleic acid(s) of interest can be pre-amplified prior to the hybridization and/or amplification step (e.g., PCR). In some cases, a nucleic acid sample may be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In some embodiments, two pairs of oligonucleotides are designed to detect a target nucleic acid as shown in FIG. 2. A T7 promoter is introduced in oligo 2 to transcribe DNA of the heteroduplex (FIG. 3). The ligated DNA fragment serves as a template for in vitro transcription reaction. The in vitro transcription reaction is carried out in the presence of T7 RNA polymerase. Optionally, the transcription reaction is carried out in the presence of a biotinylated nucleotide analog (e.g. biotin-dCTP). The labeled probes are then detected, e.g., with HRP-conjugated streptavidin and a chemulinescent substrate and thus the target nucleic acid can be measured.

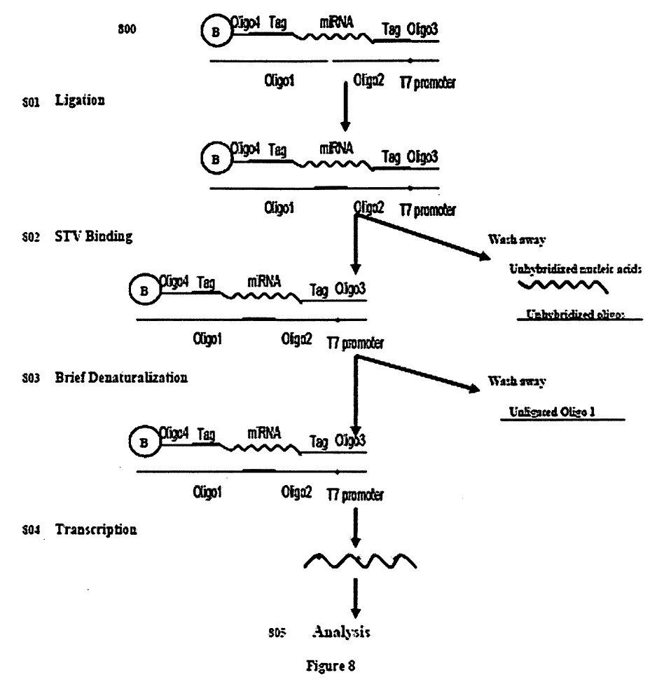

A quick overview for one of the embodiments of the invention is illustrated in FIG. 8. Even though FIG. 8 depicts a process for miRNA detection, the methods described herein can also be used in other analysis, including STR and SNP detection, RNA expression analysis, promoter methylation, gene expression, virus detection, viral subtyping and drug resistance.

A sample is obtained from a subject such as a human according to standard methods known in the art. RNA is obtained from the sample. Total RNA can be obtained from the sample using purification techniques known in the art. Generally, about 1 μg-2 μg of total RNA is sufficient. Optionally, RNA is not isolated and the miRNA is analyzed in a mixture of total DNA and RNA. In step 800 the RNA is denatured to allow the binding of oligo 1, oligo 2, oligo 3 and oligo 4. In some embodiments, oligo 4 and oligo 3 contain tags that are specific for a miRNA isoform and are complementary to sequences in oligo 1 and oligo 2. In some embodiments, the tags are part of oligo 4 and 3. In some embodiments, the tags are separate oligos that bind to oligo 1 and 2 upon denaturing and hybridization of the oligos. When the oligos are stacking together to bind to a molecule with a perfect match at the junction, it results in a specific binding to the targeted miRNA. To analyze multiple miRNAs, multiple oligo sets are mixed together, each of which is specific to one miRNA target. Each miRNA molecule will initiate the formation of RNA/DNA duplex and multiple miRNAs lead to the assembly of multiple RNA/DNA duplexes. In step 801, the stacking oligos can be ligated to form one DNA molecule. Subsequently, in step 802, the hybrids from the free oligos are separated by capturing the biotin in oligo 4 with streptavidin-conjugated structure, e.g., beads. After biotin separation, the ligated products of oligo 1 and oligo 2 are then detached from the duplexes after a brief denaturalization as depicted in step 803. In some embodiments, in order to keep the fidelity of the original miRNA abundance, PCR amplification is avoided in the preparation of hybridization probes. In step 804, the ligated fragment having the T7 promoter sequence at the 3′ end is transcribed using T7 RNA polymerase. Optionally, the transcription reaction is carried out in the presence of a biotinylated nucleotide analog (e.g. biotin-CTP). In any of the embodiments, transcription and/or amplification of ligated products may occur on a bead. In any of the embodiments herein, target nucleic acids may be obtained from a single cell. In steps 805 of FIG. 8, the amplified complementary RNA sequence is analyzed. The amplified sequence can be labeled and hybridized with a DNA microarray (e.g., 100K Set Array or other array) according to standard methods known in the art. When the transcription reaction is carried out in the presence of a biotinylated nucleotide analog, the hybridized probes can be then detected, e.g., with HRP-conjugated streptavidin and a chemulinescent substrate.

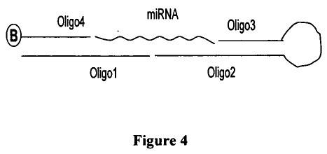

In another aspect, the invention involves a loop that links oligo 2 and oligo 3 (FIG. 4). For simplicity, this embodiment is described herein used two pairs of oligonucleotides to detect the target nucleic acids. However, the invention encompasses the use of at least 3, 4, 5, 6, 7, 8 9, 10 oligonucleotides to detect a target nucleic acid.

In a few cases target nucleic acids only differ at the 5′ end or 3′ end. These nucleic acids might anneal with the oligos that are designed for a specific target nucleic acid, and they can stay together even without ligation. This could cause false detection. In order to separate ligated molecules from hybrid molecules without ligation, a loop that links oligo 2 and oligo 3 is introduced. The ligated molecules become a perfect hairpin, which is constituted by a single molecule (FIG. 4). The hairpin duplex molecules can be separated through a capturing portion that can be introduced to the 5′ end of oligo 4 during oligo synthesis. Hairpin duplex molecules, along with all other molecules with the capturing portion can then be isolated. On example of a capturing portion that can be user is biotin. Hairpin duplex molecules, along with all other molecules with biotin will bind to streptavidin-conjugated structure, e.g., beads. After briefly denaturing, the hybrids without ligation will be dissociated and washed away. In embodiments, where the hairpin is detected by amplification, only hairpin molecules containing oligo 2 and oligo 1 sequences, would be amplified and/or transcribed by the methods describes herein since amplification primer will be designed to sequences in oligo 1 and/or oligo 2. Therefore, ligated hairpin can be detected using PCR with a pair of primers with identical sequences of ligo 3 and oligo 4.

In some embodiments, the ligated hairpin duplex molecule is amplified and optionally results are compared with amplification of similar target nucleic acids from a reference sample. Amplification can be performed by any means known in the art. In some cases, the ligated products are amplified by polymerase chain reaction (PCR). Examples of PCR techniques that can be used include, but are not limited to, quantitative PCR, quantitative fluorescent PCR (QF-PCR), multiplex fluorescent PCR (MF-PCR), real time PCR (RTPCR), single cell PCR, restriction fragment length polymorphism PCR (PCR-RFLP), PCK-RFLPIRT-PCR-IRFLP, hot start PCR, nested PCR, in situ polonony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In any of the embodiments, amplification of ligated products may occur on a bead. In any of the embodiments herein, target nucleic acids may be obtained from a single cell.

In any of the embodiments herein, the nucleic acid(s) of interest can be pre-amplified prior to the hybridization and/or amplification step (e.g., PCR). In some cases, a nucleic acid sample may be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In some embodiments, T7 promoter is introduced in oligo 2 to transcribe the hairpin heteroduplex (FIG. 4). The ligated DNA fragment serves as a template for in vitro transcription reaction. The in vitro transcription reaction is carried out in the presence of T7 RNA polymerase. Optionally, the transcription reaction is carried out in the presence of a biotinylated nucleotide analog (e.g. biotin-CTP). The hybridized probes are then detected with HRP-conjugated streptavidin and a chemulinescent substrate and thus the target nucleic acid can be measured.

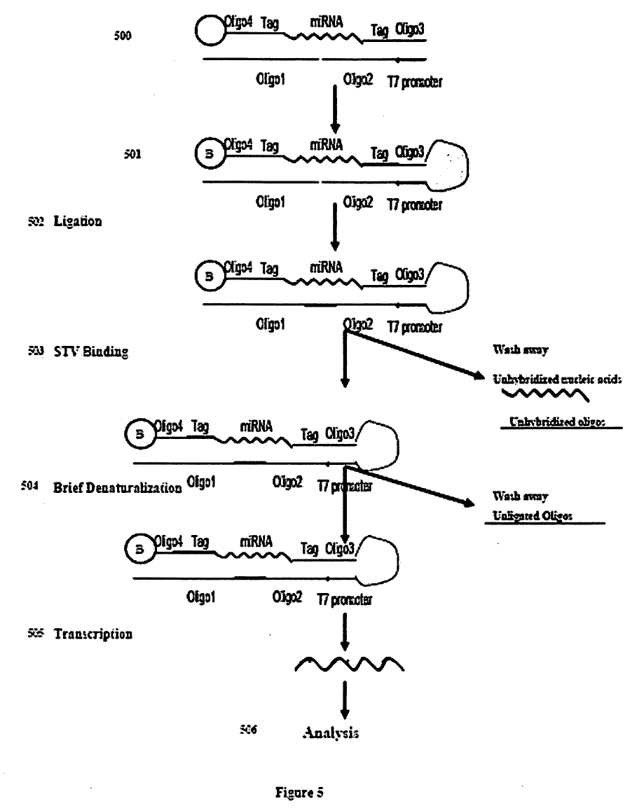

A quick overview for one of the embodiments of the invention is illustrated in FIG. 5. Even though FIG. 5 depicts a process for miRNA detection, the methods described herein can also be used in other nucleic acid analysis, including STR and SNP detection, RNA expression analysis, promoter methylation, gene expression, virus detection, viral subtyping and drug resistance.

A sample is obtained from a subject such as a human according to standard methods known in the art. RNA is obtained from the sample. Generally, about 1 μg-2 μg of total RNA is sufficient. Optionally, RNA is not isolated and the miRNA is analyzed in a mixture of total DNA and RNA. In step 500 the RNA is denature to allow the binding of oligo 1, oligo 2, oligo 3 and oligo 4. In some embodiments, oligo 4 and oligo 3 contain tags that are specific for a miRNA isoform and are complementary to sequences in oligo 1 and oligo 2. In some embodiments, the tags are part of oligo 4 and 3. In some embodiments, the tags are separate oligos that bind to oligo 1 and 2 upon denaturing and hybridization of the oligos. When the oligos are stacking together to bind to a molecule with a perfect match at the junction, it results in a specific binding to the targeted miRNA. To analyze multiple miRNAs, multiple oligo sets are mixed together, each of which is specific to one miRNA target. Each miRNA molecule will initiate the formation of RNA/DNA duplex and multiple miRNAs lead to the assembly of multiple RNA/DNA duplexes. In step 501, a loop is added that link oligo 2 and oligo 3. In step 502, the stacking oligos and the loop can be ligated to form one DNA molecule. Subsequently, in step 503, the hybrids from the free oligos are separated by capturing the biotin in oligo 4 with streptavidin-conjugated beads. In step 504, after a brief denaturing step the ligated hybrids are separated from the unligated hybrids and free oligos. In step 505, the ligated fragment having the T7 promoter sequence at the 3′ end is transcribed using T7 RNA polymerase. Optionally, the transcription reaction is carried out in the presence of a biotinylated nucleotide analog (e.g. biotin-CTP). In any of the embodiments, transcription and/or amplification of ligated products may occur on a bead. In any of the embodiments herein, target nucleic acids may be obtained from a single cell. In steps 506 of FIG. 5, the transcribed complementary RNA sequence is analyzed. The transcribed sequence can be labeled and hybridized with a DNA microarray (e.g., 100K Set Array or other array) according to standard methods known in the art. When the transcription reaction is carried out in the presence of a biotinylated nucleotide analog, the hybridized probes can be then detected, e.g., with HRP-conjugated streptavidin and a chemulinescent substrate.

### Detection

In one aspect, at least one set of oligonucleotides probes is designed to bind to a target nucleic acid. The methods described herein can be used in nucleic acid analysis including STR and SNP detection, RNA expression analysis, promoter methylation, gene expression, virus detection, viral subtyping and drug resistance.

Results can be visualized by using a label in a microtiter plate. For instance, when the transcription reaction described in FIGS. 5 and 8 is carried out in the presence of a biotinylated nucleotide analog, transcription product can be detected, e.g., with HRP-conjugated streptavidin and a chemulinescent substrate.

When analyzing target nucleic acids according to the methods described herein, the amplified and/or transcribed products of the ligated oligonucleotide probes can be labeled and hybridized with a DNA microarray (e.g., 100K Set Array or other array). Results from any of the embodiments described herein can be visualized using a scanner that enables the viewing of intensity of data collected and software to determine miRNA expression. Such methods are disclosed in part U.S. Pat. No. 6,505,125. Another method contemplated by the present invention to detect and quantify RNA expression involves the use of bead as is commercially available by Illumina, Inc. (San Diego) and as described in U.S. Pat. Nos. 7,035,740; 7,033,754; 7,025,935, 6,998,274; 6,942,968; 6,913,884; 6,890,764; 6,890,741; 6,858,394; 6,812,005; 6,770,441; 6,620,584; G,544,732; 6,429,027; 6,396,995; 6,355,431 and US Publication Application Nos. 20060019258; 0050266432; 20050244870; 20050216207; 20050181394; 20050164246; 20040224353; 20040185482; 20030198573; 20030175773; 20030003490; 20020187515; and 20020177141; and in B. E. Stranger, et al., Public Library of Science-Genetics, I (6), December 2005; Jingli Cai, et al., Stem Cells, published online Nov. 17, 2005; C. M. Schwartz, et al., Stem Cells and Development, f4, 517-534, 2005; Barnes, M., J. et al., Nucleic Acids Research, 33 (1 81, 5914-5923, October 2005; and Bibikova M, et al. Clinical Chemistry, Volume 50, No. 12, 2384-2386, December 2004. Additional description for preparing RNA for bead arrays is described in Kacharmina J E, et al., Methods Enzymol303: 3-18, 1999; Pabon C, et al., Biotechniques 31(4): 8769, 2001; Van Gelder R N, et al., Proc Natl Acad Sci USA 87: 1663-7 (1990); and Murray, S S. BMC Genetics B(SupplI):SX5 (2005).

When analyzing SNP according to the methods described herein, the amplified/transcribed products of the ligated oligonucleotide probes can be labeled and hybridized with a DNA microarray (e.g., 100K Set Array or other array). Results can be visualized using a scanner that enables the viewing of intensity of data collected and software “calls” the SNP present at each of the positions analyzed. Computer implemented methods for determining genotype using data h m mapping arrays are disclosed, for example, in Liu, et al., Bioinformatics 19:2397-2403, 2003; and Di et al., Bioinformatics 21: 1958-63, 2005. Computer implemented methods for linkage analysis using mapping array data are disclosed, for example, in Ruschendorf and Nusnberg, Bioinformatics 21:2123-5, 2005; and Leykin et al., BMC Genet. 6:7, 2005; and in U.S. Pat. No. 5,733,729.

In some embodiments of this aspect, genotyping microarrays that are used to detect SNPs can be used in combination with molecular inversion probes (MIPS) as described in Hardenbol et al., Genome Res. 15(2):269-275, 2005, Hardenbol, P. et al. Nature Biotechnology 2 1 (6), 673-8, 2003; Faham M, et al. Hum Mol. Genet. August 1; 10(16): 1657-64, 200 1: Maneesh Jain, Ph.D., et al. Genetic Engineering News V24: No. 18, 2004; and Fakhrai-Rad H, el al. Genome Res. July; 14(7):1404-12, 2004; and in U.S. Pat. No. 5,858,412. Universal tag arrays and reagent kits for performing such locus specific genotyping using panels of custom MlPs are available from Affymetrix and ParAllele. MIP technology involves the use enzymological reactions that can score up to 10,000: 20,000, 50,000; 100,000; 200,000; 500,000; 1,000,000; 2,000,000 or 5,000,000 SNPs (target nucleic acids) in a single assay. The enzymological reactions are insensitive to crossreactivity among multiple probe molecules and there is no need for pre-amplification prior to hybridization of the probe with the genomic DNA. In any of the embodiments, the target nucleic acid(s) or SNPs can be obtained from a single cell.

Another method contemplated by the present invention to detect target nucleic acids involves the use of bead arrays (e.g., such as one commercially available by Illumina, Inc.) as described in U.S. Pat. Nos. 7,040,959; 7,035,740; 7,033,754; 7,025,935, 6,998,274; 6,942,968; 6,913,884; 6,890,764; 6,890,741; 6,858,394; 6,846,460; 6,812,005; 6,770,441; 6,663,832; 5,520,584; 6,544,732; 6,429,027; 6,396,995; 6,355,431 m d US Publication Application Nos. 20060019258; 20050266432; 20050244870; 20050216207; 20050181394; 20050164246; 20040224353: 20040185482; 200 30198573; 200301 75773; 20030003490; 200201 8751 5; and 200201 77 14 1; as well as Shen, R., et al. Mutation Research 573 70-82 (2005).

In any of the embodiments of this aspect, genotyping (e.g., SNP detection) and/or quantification analysis (e.g., RNA expression) of genetic content can be accomplished by sequencing. Sequencing can be accomplished through classic Sanger sequencing methods which are well known in the art. Sequence can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read. Sequencing can be preformed using genomic DNA, cDNA derived from RNA transcripts or RNA as a template.

In some embodiments of this aspect, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. This fast sequencing method also allows for detection of a SNP nucleotide in a sequence in substantially real time or real time. Finally, SMSS is powerful because, like the MIP technology, it does not require a pre amplification step prior to hybridization. In fact, SMSS does not require any amplification. SMSS is described in part in US Publication Application Nos. 2006002471 I; 20060024678; 20060012793; 20060012784; and 20050100932.

In some embodiments of this aspect, high-throughput sequencing involves the use of technology available by 454 Lifesciences, Inc. (Branford, Conn.) such as the Pico Titer Plate device which includes a fiber optic plate that transmits chemiluninescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density pricolitre reactors”, Nature, doi: 10.1038/nature03959; and well as in US Publication Application Nos. 20020012930; 20030058629; 20030100102; 20030148344; 20040248161; 20050079510, 20050124022; and 20060078909.

In some embodiments of this aspect, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106130; 20030064398; 20030022207; and Constans, A., The Scientist 2003, 17(13):36.

In some embodiments of this aspect, high-throughput sequencing of RNA or DNA can take place using AnyDot.chjps (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO02/088382, WO03/020968, WO03/031947, WO2005/044836, PCT/EP05/105657, PCT/EP05/105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such system involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable lo move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In any of the embodiment herein of this aspect, nucleic acids can be quantified. Methods for quantifying nucleic acids are known in the art and include, but are not limited to, gas chromatography, supercritical fluid chromatography, liquid chromatography (including partition chromatography, adsorption chromatography, ion exchange chromatography, size exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, and high-throughput genotyping such as with the use of molecular inversion probe (MIP).

Quantification of amplified target nucleic acid can be used to determine gene or allele copy number, gene or exon-level expression, RNA expression, methylation-state analysis, or detect a novel transcript in order to diagnose or condition, e.g. fetal abnormality, cancer or viral infection.

Detection and/or quantification of target nucleic acids can be done using fluorescent dyes known in the art. Fluorescent dyes may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine™), carboxy tetramethylrhodamine (TAMRA™), carboxy-X-rhodamine (ROX™), LIZ™, VIC™, NED™, PET™, SYBR, PicoGreen, RiboGreen, and the like. Descriptions of fluorophores and their use, can be found in, among other places, R. Haugland, Handbook of Fluorescent Probes and Research Products, 9.sup.th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

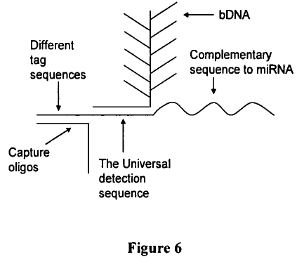

In another aspect of the invention, a branched-DNA (bDNA) approach is used to increase the detection sensitivity. In some embodiments, branched-DNA (bDNA) approach is applied to an array detection assay (FIG. 6). The array detection assay can be any array assay known in the art, including the array assays described herein. bDNA approach amplifies the signals through a branched DNA that are attached by tens or hundreds of alkaline phosphatase molecules. Thus, the signals are significantly amplified while the fidelity of the original nucleic acid target abundance is maintained.

In some embodiments, a universal detection sequence is introduced in one of the oligos describe herein. In some embodiments a universal detection sequence is introduced in oligo 1. As no labeling, e.g., biotin labeling, is required in the detection, in the embodiments where the ligated products are amplified and/or transcribed, amplification and/or transcription of the ligated product, e.g., oligo 1 and 2 can occur in the presence of regular NTPs. After hybridization via the tag sequence moieties, described herein, of the ligated products onto a substrate, (e.g. an array or beads), the universal detection sequence is then detected by bDNA. Optionally, the amplified and/or transcribed product of the ligated oligos is hybridized onto a substrate (e.g. an array or beads). Because the signals are amplified, low abundant nucleic acids and nucleic acids in limited samples can be profiled. In some embodiments, a universal detection sequence is introduced through extending the tag sequences in oligo 1 and oligo 4 (FIG. 2) or oligo 1 and oligo 5 (FIG. 7). As no labeling, e.g., biotin labeling is required in the detection, in the embodiments where the ligated products are amplified, amplification of the ligated product can occur in the presence of regular NTPs. In some embodiments, the ligation product of oligo 1 and 2 as described above is amplified by any method known in the art including those described herein. In some embodiments, the hairpin product described above is amplified by any method known in the art including those described herein. After hybridization via the tag sequence moieties of the amplified nucleic acids onto a substrate (e.g. beads or array), the universal detection sequence is then detected by bDNA. Because the signals are amplified, low abundant nucleic acids and nucleic acids in limited samples can be profiled.

### Heterogenous Annealing and Ligation

In one aspect of the invention, instead of performing the assay with soluble probes and thereafter immobilizing, one of the probes or the target nucleic acid may be immobilized on a solid support prior to annealing. In some embodiments, when one of these probes is immobilized, one of the other probes is labeled and in solution phase. This permits detection of label immobilized to the solid support based on the ligation. In some embodiments, when the target nucleic acid is immobilized, both the labeled and unlabeled probes are soluble in the fluid medium.

Techniques to immobilize nucleic acids, including the probes of the present invention, onto solid supports such as commercially available polymers, nylon, nitrocellulose membranes and dextran supports or beads are well known to those skilled in the art. Other immobilization techniques include attachment of biotinylated probes to immobilized streptavidin, the linking of amino groups on the probe to amino groups on a membrane bound protein support via a bifunctional linking reagent such as disuccinimidyl suberate and the methods described by Bischoff, et al. (1987), Anal. Biochem., 164, 336; Goldkorn, et al. (1986), Nucl. Acids Res., 14, 9171; Jablonski, et al. supra and Ghosh F., et al. (1987) Anal Biochem, 164, 336-344. Thus, for example, in one embodiment an adjacent probe may be bound to a solid support and contacted with a target nucleic acid under conditions which permit annealing of the adjacent probe to the complementary region of the target nucleic acid in a sample. Thereafter (or simultaneously therewith) the other probe(s) is contacted with the target nucleic acid to permit annealing of the target probe with the test DNA region immediately adjacent and contiguous to the adjacent probe. In some embodiments, one of the soluble probes contains a label. If necessary, the temperature is adjusted to maintain enzymatic activity of T4 DNA ligase which is thereafter contacted with the annealed target and adjacent probes to produce ligation if base pair matching in the end region of the target probe is present. Thereafter, the stringency of the fluid medium is raised to remove substantially all the species of the probes which are not ligated to the adjacent probe and/or target nucleic acid. The ligated product is then detected by standard techniques by measuring the ligated product bound to the solid support.

Alternatively, a biotinylated probe can be immobilized on a streptavidin-coated solid support (e.g., agarose beads).

The biotin-streptavidin binding phenomenon (or for that matter, any other binding phenomenon such as antibody-antigen binding, etc.) may also be utilized in a modified heterogenous assay. Thus, for example, one of the probes may be immobilized on a solid support by standard techniques. A biotinylated soluble probe is then employed in the assay as described. If ligation occurs the biotinylated ligated product will be bound to the solid support. Thereafter, any label linked to streptavidin, e.g., radioisotope, enzyme, etc. is contacted with the immobilized biotinylated linked probe product and assayed using standard techniques to ascertain whether the ligation event occurred.

It is also possible to assay for more than one target nucleic acid by using immobilized probes. Thus, sets of probes as described above each specific for one target nucleic acid may be employed. In some embodiments, each of the unlabeled probes from each probe set is immobilized in physically discrete sections on a solid support. In this manner, each discrete location represents a separate test for a particular target nucleic acid. Thereafter, the target nucleic acid is contacted with each of the immobilized probes. A mixture containing probes from each of the probe sets as described above is added. In some embodiment, a mixture containing labeled soluble probes from each of the above probe sets is then added. Each of these soluble probes is capable of annealing to the target nucleic acid and/or other probes in continuity with the immobilized probe. After ligation (if it occurs), non-ligated probes are removed from the solid support and ligated probe products immobilized on the solid support is detected. The detection of a ligated probe product in a particular discrete location on the support provides an indication of the presence or absence of the target nucleic acid.

Instead of immobilizing one of the probes, the target nucleic acid may also be immobilized to a solid support. Thus, for example, the target nucleic acid is transferred to, e.g., a nitrocellulose, nylon membrane or a bead by standard techniques.

### Kits

In an embodiment, a kit is provided for a detection and/or quantitation of a target nucleic acid. The kit includes: an oligo mix containing the oligonucleotide probes described herein. In addition, kits are provided which comprise reagents and instructions for performing methods of the present invention, or for performing tests or assays utilizing any of the compositions, arrays, or assemblies of articles of the present invention. The kits may further comprise buffers, restriction enzymes, adaptors, primers, a ligase, a polymerase, dNTPS, NTPs, detection reagents and instructions necessary for use of the kits, optionally including troubleshooting information.

### Methods

The methods described herein discriminate between nucleotide sequences. The difference between the target nucleotide sequences can be, for example, a single nucleic acid base difference, a nucleic acid deletion, a nucleic acid insertion, or rearrangement. Such sequence differences involving more than one base can also be detected. In some embodiments, the oligonucleotide probe sets have substantially the same length so that they hybridize to target nucleotide sequences at substantially similar hybridization conditions. As a result, the process of the present invention is able to detect infectious diseases, genetic diseases, and cancer. It is also useful in environmental monitoring, forensics, and food science. Examples of genetic analyses that can be performed on nucleic acids include e-g., SNP detection, STR detection, RNA expression analysis, promoter methylation, gene expression, virus detection, viral subtyping and drug resistance.

A wide variety of infectious diseases can be detected by the process of the present invention. Typically, these are caused by bacterial, viral, parasite, and fungal infectious agents. The resistance of various infectious agents to drugs can also be determined using the present invention.

Bacterial infectious agents which can be detected by the present invention include Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Fungal infectious agents which can be detected by the present invention include Cryptococcus neoformans, Blastomyces dermatitidis, Histoplasma capsulatum, Coccidioides immitis, Paracoccidioides brasiliensis, Candida albicans, Aspergillus fumigautus, Phycomycetes (Rhizopus), Sporothrix schenckii, Chromomycosis, and Maduromycosis.

Viral infectious agents which can be detected by the present invention include human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Parasitic agents which can be detected by the present invention include Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention is also useful for detection of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can all be identified with the present invention.

Genetic diseases can also be detected by the process of the present invention. This can be carried out by prenatal or post-natal screening for chromosomal and genetic aberrations or for genetic diseases. Examples of detectable genetic diseases include: 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Disease, thalassemia, Klinefelter Syndrome, Huntington Disease, autoimmune diseases, lipidosis, obesity defects, hemophilia, inborn errors of metabolism, and diabetes.

Cancers which can be detected by the process of the present invention generally involve oncogenes, tumor suppressor genes, or genes involved in DNA amplification, replication, recombination, or repair. Examples of these include: BRCA1 gene, p53 gene, APC gene, Her2/Neu amplification, Bcr/Ab1, K-ras gene, and human papillomavirus Types 16 and 18. Various aspects of the present invention can be used to identify amplifications, large deletions as well as point mutations and small deletions/insertions of the above genes in the following common human cancers: leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms.

In the area of environmental monitoring, the present invention can be used for detection, identification, and monitoring of pathogenic and indigenous microorganisms in natural and engineered ecosystems and microcosms such as in municipal waste water purification systems and water reservoirs or in polluted areas undergoing bioremediation. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can also be used in a variety of forensic areas, including for human identification for military personnel and criminal investigation, paternity testing and family relation analysis, HLA compatibility typing, and screening blood, sperm, or transplantation organs for contamination.

In the food and feed industry, the present invention has a wide variety of applications. For example, it can be used for identification and characterization of production organisms such as yeast for production of beer, wine, cheese, yoghurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

### EXAMPLES

### Example 1

### Sample Preparation

Nucleic acids samples can be obtained from any tissue according to standard techniques known in the art.

a. miRNA Analysis

miRNA samples can be obtained from any tissue according to standard techniques known in the art. For instance, samples can be obtained from blood. For instance, miRNA samples can be obtained from white blood cells. Briefly, blood from a subject can be obtained in EDTA-containing blood collection tubes. Red blood cells are lysed by mixing the blood samples with 4 volumes of lysis buffer (10 mM Tris pH 8.0, 10 mM EDTA). After 10 min on ice with occasional agitation, the suspensions are centrifuged and the supernatants are decanted. The white blood cell pellets are resuspended in 20 ml of lysis buffer, and the above process is repeated. The white blood cells are then first lysed in a denaturing lysis solution which stabilizes RNA and inactivates RNases. The lysate is then extracted once with Acid Phenol:Chloroform which removes most of the other cellular components, leaving a semi-pure RNA sample.

Some of the methods describe herein do not need further purification of miRNA. However, in some embodiment a further isolation step may be performed. In order to perform this purification, the sample from above can be further purified according to standard techniques known in the art. For instance, the samples above can be further purified over a glass-fiber filter by one of two protocols from Ambion's mirVana™ miRNA isolation kit according to the manufacturer instructions to yield either total RNA or a size fraction enriched in miRNAs.

b. Genomic DNA Preparation

Genomic DNA samples can be obtained from any tissue according to standard techniques known in the art. For instance, samples can be obtained from blood. Genomic DNA can be prepared from the blood of subjects according to standard techniques known in the art. Briefly, blood can be obtained in EDTA-containing blood collection tubes. Red blood cells are lysed by mixing the blood samples with 4 volumes of lysis buffer (10 mM Tris pH 8.0, 10 mM EDTA). After 10 min on ice with occasional agitation, the suspensions are centrifuged and the supernatants are decanted. The white blood cell pellets are resuspended in 20 ml of lysis buffer, and the above process is repeated. Each cell pellet is then suspended in 15 ml of digestion buffer (50 mM Tris pH 8.0, 5 mM EDTA, 100 mM NaCl, 1% SDS) and 3 mg (0.2 mg/ml) of proteinase K is added. The cells are digested at 37° C. for 5 hours. The digests are extracted twice with equal volumes of phenol, then once with equal volumes of a 1:1 phenol:chloroform mixture and finally once with equal volumes of chloroform, each time centrifuging the mixture and removing the aqueous phase for the next extraction. After the final extraction and removing the aqueous phases, one tenth volume of 3 M sodium acetate, pH 6.5, is added. Two volumes of ice cold 100% EtOH are then added to each solution to precipitate the genomic DNAs, which are spooled out of solution on glass pipettes. The DNA precipitates are washed twice in 0.75 ml volumes of 70% EtOH, briefly centrifuging each time to allow removal of the supernatants. After removing the supernatants for the second time, the remaining EtOH is allowed to evaporate and the DNA is suspended in 0.5 ml of TE (10 mM Tri-HCl pH 8.0 containing 1 mM EDTA) solution. A fifth dilution of each DNA solution is also prepared in TE.

To determine the concentrations of the one fifth DNA solutions can be determined according to standard techniques known in the art.

To digest the genomic DNAs with Taq I, 25 μl of the 100 ng/μl solutions is mixed with 5 μl of 10× medium salt buffer (0.5 M NaCl, 0.1 M MgCl₂, 0.1 M Tris, pH 8.0), 20 μl of water-ME (i.e. water containing 6 mM ME (i.e., mercaptoethanol)), and 400 U of Taq I restriction endonuclease. The digests are covered with mineral oil and incubated at 65° C. for 1 hour. The reactions are stopped by adding 1.2 μl of 500 mM EDTA and heating the specimens to 85° C. for 10 min. Complete digestion of the DNAs is checked by electrophoresing aliquots on a 1% agarose gel.

### Example 2

### Oligonucleotide Preparation

Oligonucleotides can be synthesized according to standard techniques known in the art. For instance, oligonucleotides can be synthesized on a 394A DNA Synthesizer (Applied Biosystems Division of Perkin-Elmer Corp., Foster City, Calif.). Oligonucleotides labeled with Biotin can be synthesized using the manufacturer's suggested modifications to the synthesis cycle (Applied Biosystems Inc., 1994).

OLA oligonucleotides are purified by ethanol precipitation after overnight deprotection at 55° C. The primer-specific portions of the oligonucleotides used for PCR amplification are purified by polyacrylamide gel electrophoresis on 10% acrylamide/7M urea gels. Oligonucleotides are visualized after electrophoresis by UV shadowing against a lightening screen and excised from the gel (Applied Biosystems Inc., 1992). They are then eluted overnight at 64° C. in TNE (i.e. Tris-sodium EDTA) buffer (100 mM Tris/HCl pH 8.0 containing 500 mM NaCl and 5 mM EDTA) and recovered from the eluate using Sep Pak cartridges (Millipore Corp, Milford, Mass.) following the manufacture's instructions.

Oligonucleotides are resuspended in 100 μl TE (i.e. 10 mM Tri-HCl pH 8.0 containing 1 mM EDTA). Typical concentrations of these original OLA probe solutions are about 1 μg/μl or approximately 74 pm/μl.

As a prerequisite for the OLA phase, the downstream OLA oligonucleotides probes are phosphorylated with T4 polynucleotide kinase. Aliquots of the 5 downstream oligonucleotides equivalent to 200 pm are combined with 10 μl of 10× kinase buffer (500 mM Tris/HCl pH 8.0, 100 mM MgCl₂), 10 μl of 10 mM ATP, 20 U T4 kinase, and sufficient water-ME to give a final volume of 100 μl. Phosphorylation is carried out at 37° C. for 30 min followed by incubation for 10 min at 85° C. to inactivate the T4 enzyme.

The solutions of the OLA and PCR oligonucleotides are adjusted to convenient concentrations. The kinased OLA probe solution is diluted fourfold in water to yield a concentration of 1000 fm/μl. A solution of the upstream OLA probes is made by combining volumes of the probes equivalent to 200 pm with sufficient water to give a final volume of 400 μl. This created a solution 1000 fm/μl in each of the upstream OLA probes. Aliquots (20 μl) of the kinased and unkinased OLA probes are frozen for subsequent use.

Branched oligonucleotides can be synthesized according to any standard techniques known in the art. Branched oligonucleotides can be synthesized by chemical cross-linking of oligonucleotides containing three alkylamine functions as described in Clinical Chemistry (1993), 39(4): 725. Alternatively, branched oligonucleotides can be produced by incorporating “branching” monomers” during the chemical synthesis of oligodeoxyribonucleotides (Clinical Chemistry (1993), 39(4): 725). BMs are phosphoramidite reagents containing at least two protected hydroxyl functions. In general, a primary linear fragment is synthesized and then tailed with several appropriately spaced BMs. Several simultaneous secondary syntheses are then conducted from the branch points. Branched oligonucleotides containing several hundred nucleotides can be constructed in this way. Large-branched oligonucleotides for signal amplification can be synthesized by using a combination of solidphase chemistry and enzymatic ligation methods. For instance, an amplifier containing a maximum of 45 alkaline phosphatase probe-binding sites can be produced (1068 nucleotides). It can be constructed by synthesizing a bDNA with 15 branches (168 bases), which is then combined with a complementary linker that is in turn complementary to a branch extension (or “arm”; 60 bases), each of which has three binding sites for an alkaline phosphatase probe to bind (three sites times 15 branches=45 labels). The amplifiers are assembled by treatment with T4 DNA ligase, then analyzed by capillary electrophoresis.

FIGS. 1 to 4 show the design of OLA oligonucleotide probes for detection and quantification of miRNA in an OLA/PCR process. However, the oligonucleotides probes described herein can be use to determine any target nucleic acid of interested. In FIGS. 1 to 4, these oligonucleotides are designed to specifically detect a single miRNA molecule. A pair of oligos is designed and synthesized, oligo 1 and oligo 2, to correspond to one miRNA molecule. Oligo 2 will have a phosphate group at its 5′ end. When these two oligos simultaneously bind to one miRNA molecule, they are ligated by T4 DNA ligase (FIG. 1). One of the oligos may non-specifically bind to a RNA or DNA molecule, but it would not result in detection, as these non-specific bindings of the oligos along with free oligos will be eliminated or removed by a separation as described below. When two oligos are stacking together to bind to a molecule with a perfect match at the junction, it results in a specific binding to the targeted miRNA. The stacking oligos can be ligated to form one DNA molecule, which can be used for detection. Any sequence-closely related miRNA molecules will either block the ligation or prevent the hybrid formation. Therefore, isoforms can be distinguished in the assay. If the difference is in the middle of the miRNA, it will block the ligation and detection, although the hybrids are able to form.

Because miRNA precursors contain the identical sequence of a mature miRNA, they can be targeted by the pair oligos, leading to ligation and detection. In order to exclude miRNA precursors from the detection oligo 1 and 2, with two unique tag sequences, are extended corresponding to the sequence of oligo 3 and 4. These oligos form two partial duplexes with a protruding sequence, each of which is able to hybrid a part of a target miRNA molecule. If the target is a mature miRNA molecule, the hybridization forms two perfect matches with oligo 3 and oligo 4 at the ends of the miRNA (FIG. 2), leading to a ligation. If the target is a precursor miRNA, no perfect match ends are forming at the junctions between the precursor and oligo 3 or/and between the precursor and oligo 4, and therefore no ligation can occur and no detection can be made. Furthermore, any nucleotide difference that exists at the ends among miRNA isoforms will result in imperfect match, which blocks either the formation of a hybrid or the ligation.

To analyze multiple miRNAs, e.g., in an array analysis, multiple oligo sets are mixed together, each of which is specific to one miRNA target. Each miRNA molecule will initiate the formation of RNA/DNA duplex and multiple miRNAs lead to the assembly of multiple RNA/DNA duplexes.

If array analysis is used to detect the miRNA target, the isoforms of a miRNA with one single nucleotide difference will be very difficult to be distinguished by array hybridization if miRNA sequences are directly used for spotting. To distinguish them, a unique tag sequence is assigned in oligo 3 oligo 4 for each isoform, depending on the location of the different nucleotide. These tag sequences becomes new markers for miRNAs, which can be easily differentiated on array.

To separate the hybrids from the free oligos, a biotin at oligo 4 is introduced, which can be captured by streptavidin-conjugated beads. After biotin separation, the ligated products of oligo 1 and oligo 2 are then detached from the duplexes.

In order to keep the fidelity of the original miRNA abundance, PCR amplification is avoided in the preparation of hybridization probes. A T7 promoter is introduced in oligo 2 to transcribe DNA of the heteroduplex (FIG. 3). The transcription will be carried out in the presence of biotin-CTP and the transcribed RNA used as the probe for array hybridization. The hybridized probes are then detected with HRP-conjugated streptavidin and a chemulinescent substrate and thus miRNAs can be measured. A specific miRNA or isoform can be identified and differentiated according to the tag sequence, e.g., by the position of its corresponding tag sequence on an array or by sequencing the transcription product. Therefore, high discrimination array analysis of all miRNAs.

In a few cases, isoforms that only differ at the 5′ end or 3′ end. They might anneal with the oligos that are designed for a specific mature miRNA molecule, and they can stay together even without ligation. This could cause false detection. In order to separate ligated molecules from hybrid molecules without ligation, a loop that links oligo 2 and oligo 3 is introduced. The ligated molecules become a perfect hairpin, which is constituted by a single molecule (FIG. 4). The hairpin miRNA/DNA duplex molecules can be separated through a biotin that will be introduced to the 5′ end of oligo 4 during oligo synthesis. Hairpin miRNA/DNA molecules, along with all other molecules with biotin will bind to streptavidin-conjugated beads. After briefly denaturing, the hybrids without ligation will be dissociated and washed away. Among the molecules that stay on the column, only hairpin molecules contain oligo 2 and oligo 1 sequences, both of which are required for PCR. Therefore, ligated hairpin can be detected using PCR with a pair of primers with identical sequences of ligo3 and oligo 4.

### Example 3

### T7-OLA Process

Materials: Oligo Mix (200 fmol/each target), Hybridization buffer, Streptavidin magnetic beads (Fisher), Beads washing buffer, ligase, ligation buffer (Femantas), Pre-reaction buffer, NTP mix (Roche), 10×T7 transcription buffer, T7 RNA transcriptase, Hybridization buffer, Hybridization washing solution, 1× Blocking buffer, Streptavidin-HRP conjugate, Washing buffer, Luminol/Enhancer Solution, Stable Peroxide Solution, Magentic stand (96 well plate or 24 well stand), PCR machine (for example. MJ), Hybridization oven, Washing tray, 0.2 ml or 0.4 ml tubes, Alpha Innotech image or equivalent image system or X-ray film.

Hybridization of miRNA with Oligos:

a. Sample Preparation

From cultured cell lysate: Add 1 ml of cell lysate buffer per 1-2×10⁵ cells, and heat at 100° C. for 5 minutes and cool on ice, 80 μl is used for assay. From total RNA or DNA: Add 70 μl to 10 μl 100 ng-1 μg RNA or DNA, and heat at 100° C. for 5 minutes and cool on ice.

Incubate RNA or DNA sample with oligo mix through mixing the following components: 80 μl sample, 3 μl oligo mix, 2 μl of oligo mix 2, 15 μl hybridization buffer (500 mM NaCl, 20 mM Tris.HCl, 5 mMEDTA).

Incubate on PCR machine at 94° C. for 2 minutes, 55° C. for 10 minutes, and 35° C. for 1 hour

Selection of miRNA/Oligo Hybrids:

a. Washing Beads

Add 5 μl beads with 150 μl of hybridization buffer in a tube, the size of the tube that should fit into the magnetic stand. Stay on the magnetic stand for 40 seconds. Aspirate out the liquid. Take out the tube from magnetic stand and add hybridization buffer, repeat one more time.

b. Beads Selection

Add 100 μl oligo mixture to the washed beads and resuspend the beads in solution. Incubate for 30 minutes. Put the bead mixture on the magnetic stand and stay for 30 second, and aspirate out the buffer. The beads remain on the side of tube. Remove the tube from the magnetic stand and add 150 μl of bead washing buffer (100 mM NaCl, 10 mMTris, Hcl, pH7.2, 5 mM EDTA, 0.1% Tween-20). Repeat the washing step for two times.

Ligation of miRNA-directed pairing oligos to form a single molecule: The procedure is following to manufacturer's instruction. Add 50 μl of ligation buffer and put the tube on the magnetic stand for 30 seconds, remove the buffer. Add 1 μl ligase in 40 μl ligation buffer to make ligation mixture, completely resuspend the beads with ligation mixture. Incubate at room temperature for 1 hour.

T7 RNA transcription of ligated molecule: Add 201 of pre-reaction buffer to resuspend the beads. Incubate the mixture at 94° C. for 45 second, 55° C. for 30 second and 68° C. for 45 second. Put the reaction tube on the magnetic stand for 30 second. Transfer the 20 μl of reaction buffer to a fresh tube, and add 20 μl T7 RNA polymerase mixture containing: (i) 4 μl 5×T7 transcription buffer, (ii) 4 μl NTP mixture, (iii) 1 μl T7 RNA polymerase and (iv) 11 μl ddH₂O. Incubate at 37° C. for 1 hour. The reaction mixture is ready for further analysis.

### Example 4

### Array Membrane

The reaction mixture of Example 3 can be analyzed using an array membrane containing probes design to hybridize with the nucleic acids produced in the reaction mixture of Example 3 consistent with the methods described herein.

Pre-hybridization and hybridization: Place each array membrane into 50 ml tube. Wet the membrane by filling the tube with dH₂O, then carefully decant the water. The side of the membrane with the spotted oligos should face into the middle of the tube. Add 3-5 ml of prewarmed Hybridization Buffer to each tube. Incubate the tubes in a hybridization oven at 42° C. for 1 hour. Add 40 μl T7 transcript products to prehybridized membrane and incubate overnight in a hybridization oven. Decant the hybridization mixture from each bottle and wash each membrane as follows: (i) Fill each bottle with 30 ml Hybridization Washing Solution, rinse the tube, and decant liquid, (ii) Fill each bottle with 30 ml Hybridization Washing Solution and incubate in oven for 20 minutes. Decant liquid.

Detection: Using forceps, carefully remove each membrane from the hybridization tube and transfer to a new container (an empty 200 μl pipette tip box). Each box could have two membranes, one at each side of the box. Rinse with Washing Buffer. Block the membrane with 15 ml of 1× Blocking Buffer for 30 minutes (at room temperature with gentle shaking for this step and following). Dilute 15 μl of Streptavidin-HRP conjugate into 1 ml of Blocking Buffer and add to box. Do not add it directly onto the membrane. Decant the Blocking Buffer and wash three times at room temperature with 1× Wash Buffer, 5 minutes each wash. Add 20 ml of 1× Detection Buffer to each membrane and incubate for 5 minutes. Combine equal amounts of Stable Peroxide Solution and Luminol/Enhancer Solution. Place the membrane on a plastic sheet protector or overhead transparency. Overlay each membrane with 1 ml of substrate solution, ensuring that the substrate is evenly distributed over the membrane. Place another plastic sheet over the top of the membrane, without trapping air bubbles on the membrane. Incubate at room temperature for 5 minutes. Remove excess substrate by pressing a paper towel over the plastic sheet. Expose the membranes using either Hyperfilm ECL (2-10 min) or a chemiluminescence imaging system (i.e., FluorChem imager from Alpha Innotech). With either method, experiment with different exposure times. Use Table 1 as a schematic diagram of human miRNA array I (shown below) to identify the spots on the array.

TABLE 1
Schematic diagram of human miRNA array I

| Let-7a | Let-7b | Let-7c | Let-7d | Let-7e | Let-7f | Let-7g | Let-7i | miR-1 | miR-7 |
|---|---|---|---|---|---|---|---|---|---|
| miR-9 | miR-10a | miR-15a | miR-15b | miR-16 | miR-17-5p | miR-18a | miR-18b | miR-19a | miR-19b |
| miR-20a | miR-21 | miR-25 | miR-28 | miR-34a | miR-99a | miR-122a | miR-124a | miR-125a | miR-125b |
| miR-126 | miR-131 | miR-133a | miR-133b | miR-143 | miR-145 | miR-146a | miR-146b | miR-148a | miR-155 |
| miR-181a | miR-181b | miR-181c | miR-182 | miR-192 | miR-194 | miR-195 | miR-199a | miR-199b | miR-199a* |
| miR-200a | miR-200c | miR-204 | miR-206 | miR-216 | miR-223 | miR-224 | miR-342 | miR-368 | miR-375 |

### Example 5

### Discrimination of Isoforms of let7 miRNA with miRNA Microarray

New discovered microRNAs (miRNAs) are single-stranded RNAs usually approximately 22 nt long. miRNA are important to the regulation of gene expression. These small molecules inhibit protein production through selective binding to the complementary messenger RNA sequences. Although the inhibition-mediated biological function of these miRNA molecules are not yet fully understood, miRNAs seems to be crucial in diverse regulations, including development, cell differentiation, proliferation, apoptosis, and maintenance of stemness and imprinting.

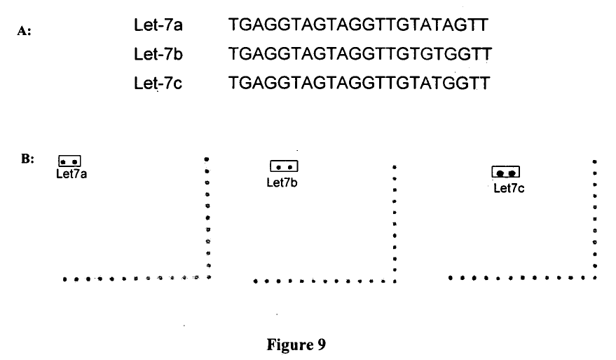

Many miRNA have been identified through both biological approach and informatics analysis. To date, there are total 475 human miRNA genes listed in the miRNA database (http://microrna.sanger.ac.uk/sequences/ftp.shtml) and it is expected to be approximately 1000, which would be equivalent to almost 3% of the protein-coding genes. Many of mature human miRNAs are closely related in sequences and more than 20% are grouped into isoforms with nearly identical sequences, usually differing by 1-3 nt. The largest human isoform families include let-7, including 9 mature molecules with different sequences. These families are designated with a letter (e.g. let-7b and let-7c). Because of the minor difference of isoforms in addition to the small size of the molecules and coexistence with precursors, it is quite challenge to analyze or profile miRNAs. FIG. 9A shows the sequences of let7a, let7b and let7c. These isoforms are closely related in sequences. The sequence differences are highlighted with red (see FIG. 9A).

To determine whether the probes described herein are able to distinguish between the different let-7 isoforms, 1 fmol of synthetic miRNAs of let-7a, let-7b and let-7c were annealed with primer pools which contain oligos for detection of 60 miRNAs. The synthetic miRNAs and the oligos were allowed to form specific miRNA/oligo hybrids as described above. After selection, the hybrids were ligated to become single molecules. The ligated DNAs were amplified and biotin labeled by T7 transcription procedure described in Example 3 and 4. The products were hybridized with miRNA array. FIG. 9B shows the results of the array hybridization. FIG. 9B shows that the specific isoform can be distinguished with the array analysis.

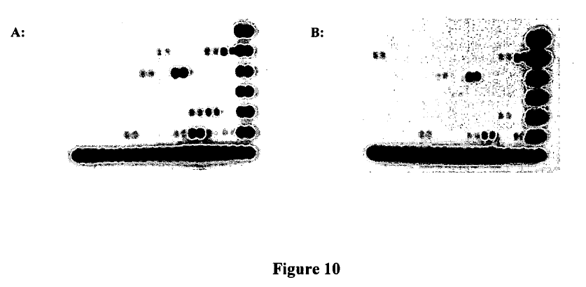

In order to determine whether the probes described herein are able to distinguish between the different let-7 isoforms from a nucleic acid sample isolated from a cell, 100 ng of HeLa RNA was incubated with primer pools which contain oligos for detection of 60 miRNAs. The miRNA and oligos were allowed to form specific miRNA/oligo hybrids as described above. After selection, the target specific hybrids were ligated to single molecules. The single molecules were then amplified and biotin labeled with PCR (see FIG. 10A), and T7 transcription (see FIG. 10B) respectively

### Example 6

### Bead Array Analysis

The arrays are spotted in triplicate, contain controls for monitoring hybridization specificity, include dye normalization controls, and have positive and negative controls spotted throughout the array.

After the OLA reactions, and amplification and/or transcription of the products, readout of the nucleic acid types can be done using arrays as described in Gunderson et al. Nature Genetics 37(5) 549-554, (2005). Oligonucleotide probes on the array are specific for the target nucleic acid, e.g. miRNA, and for the OLA probes. For, instance, the oligonucleotides can be 38 to 50 bases in length, 15 bases at the 5′ end and 3′ end for decoding and the remaining 20 bases are nucleic acid specific. The oligonucleotides are immobilized on activated beads using a 5′ amino group.

The amplification products of the OLA reaction are denatured at 95° C. for 5 min and then exposed to the Sentrix array matrix, which is mated to a microtiter plate, submerging the fiber bundles in 15 ml of hybridization sample. The entire assembly is incubated for 14-18 h at 48° C. with shaking. After hybridization, arrays are washed in 1× hybridization buffer and 20% formamide at 48° C. for 5 min.

For amplification where biotin-dCTP is used, the biotin-labeled nucleotides incorporated during amplification are then detected as described in Pinkel et al. PNAS 83 (1986) 2934-2938. The arrays are blocked at room temperature for 10 min in 1 mg ml⁻¹ bovine serum albumin in 1× hybridization buffer and then washed for 1 min in 1× hybridization buffer. The arrays are then stained with streptavidin-phycoerythrin solution (1× hybridization buffer, 3 μg ml⁻¹ streptavidin-phycoerythrin (Molecular Probes) and 1 mg ml⁻¹ bovine serum albumin) for 10 min at room temperature. The arrays are washed with 1× hybridization buffer for 1 min and then counterstained them with an antibody reagent (10 mg ml⁻¹ biotinylated antibody to streptavidin (Vector Labs) in 1×PBST (137 mM NaCl, 2.7 mM KCl, 4.3 mM sodium phosphate, 1.4 mM potassium phosphate and 0.1% Tween-20) supplemented with 6 mg ml⁻¹ goat normal serum) for 20 min. After counterstaining, the arrays are washed in 1× hybridization buffer and restained them with streptavidin-phycoerythrin solution for 10 min. The arrays are washed one final time in 1× hybridization buffer before imaging them in 1× hybridization buffer on a custom CCD-based BeadArray imaging system. The intensities are extracted intensities using custom image analysis software.

### Example 7

### Micro Array Analysis

Oligonucleotide probes on the array are specific for the target nucleic acid, e.g. miRNA, and for the OLA probes. For, instance, the oligonucleotides can be 38 to 50 bases in length, ˜15 bases at the 5′ end and 3′ end for decoding and the remaining 20 bases are nucleic acid specific. The oligonucleotides are immobilized on activated beads using a 5′ amino group. 5′ Amine oligonucleotides were resuspended in 1× Micro Spotting Plus buffer (ArrayIt, Sunnyvale, Calif.) at 20 μM concentration. Each oligonucleotide probe is printed four times on CodeLink-activated slides (GE health/Amersham Biosciences, Piscataway, N.J.) by a Pixsys7000 pin-based dispensing system (Genomics Solutions, Irvine, Calif.) in 2×2 pin and 40×8 spot configuration of each sub-array, with a spot diameter of 120 pm. The printed slides are further processed according to the manufacturer's recommendations. The array can also contains several 23 bp U6 and Drosophila tRNA oligonucleotides specifically designed as labeling and hybridization controls (positive) while 23 bp random oligonucleotides are designed as negative controls.

Hybridization buffer consists of 100 mM 2-(N-morpholino)ethanesulfonicacid (MES), 1 M [Na+], 20 mM EDTA, 0.01% Tween-20, and 0.5 mg/ml acetylated BSA. Target hybridization is done at 45° C. for 16 h, and slides are washed four times (6 min each) in buffer A (6×SSPE and 0.01% Tween-20) at RT, and then twice with buffer B (100 mM MES, 0.1 M [Na+] and 0.01% Tween-20) for 8 min at 45° C. Slides are then incubated for staining with Streptavidin solution mixture (100 mM MES, 1 M [Na+], 0.05% Tween-20, 2 mg/ml BSA and 10 μg/ml R-Phycoerythrin streptavidin) from Invitrogen at RT for 10 min followed by four washes with buffer A (6 min each) at 30° C.

Second staining is carried out with antibody solutions (100 mM MES, 1 M [Na+], 0.05% Tween-20, 2 mg/ml BSA, 0.1 mg/ml goat IgG and 5 μg/ml biotin anti-streptavidin) at RT for 10 min followed by washing with buffer A (twice) for 4 min. Third staining is performed with Streptavidin solution mixture at RT for 10 min and slides are washed four times (6 min each) with wash buffer A at 30° C. Finally, slides are washed one time, 5 min each at RT with 0.2×SSC and followed by a similar wash with 0.1×SSC to remove any salt remnant and binding particles to the slides.

### Example 8

### bDNA Analysis

Because a few of biotins are labeled on each probe and the templates for preparing probes are not amplified, the detection sensitivity is expected to be low and therefore this approach is not appropriate to profile those low abundant miRNAs or miRNAs in limited samples. To increase the detection sensitivity, a branched-DNA (bDNA) approach in the array detection (FIG. 6) can be used. Instead of template amplification like PCR, it amplifies the signals through a branched DNA that are attached by tens or hundreds of alkaline phosphatase molecules. Thus, the signals are significantly amplified while the fidelity of the original target nucleic acid abundance is maintained. First a universal detection sequence is introduced through extending the tag sequences in oligo 1 and oligo 4 (FIG. 7). As no biotin labeling is required in the detection, transcription can take place in the presence of regular NTPs. After hybridization via the tag sequence moieties of the amplification products of the OLA reaction onto the array, the universal detection sequence is then detected by bDNA. Because the signals are amplified, low abundant nucleic acids, e.g., low abundant miRNAs and miRNA in limited samples, can be profiled.

The bDNA can then used in a solution-phase sandwich assay (see FIG. 6). The amplification products of the OLA reaction are denatured and hybridized in solution to two sets of oligonucleotide probes: the capturing probes with extensions and the labeling probes. Once the probe-target complex is bound to the well of the microtiter dish, the well is washed. The bDNA is then hybridized. After a wash, the bDNA is labeled with an alkaline phosphatase probe (18 bases). Finally, the complex is detected with a dioxetane substrate that can be triggered by an enzyme, (Lumigen, Detroit, Mich.) yielding a chemiluminescent output detectable with a luminometer.

bDNA assay procedure. Capture of the OLA/PCR products on the microwell surface is accomplished by adding 200-μl aliquots of each OLA/PCR product to the appropriate oligonucleotide-modified microwell. For the standard curve which is run on every assay plate, 50-μl aliquots of standards are added to the appropriate wells on the same microplate. The microplate then is sealed with high-density polyethylene sheets under silicon pads and incubated overnight (12 to 16 h) at 53° C. in a microwell plate heater (Chiron Corporation). The microwells are allowed to cool at room temperature for 10 min and then washed twice with wash A (0.13 SSC [13 SSC is 0.15 M sodium chloride plus 0.015 M sodium citrate], 0.1% sodium dodecyl sulfate). After incubation at 53° C. for 30 min with a 50-μl volume of preamplifier/amplifier diluent (prepare by incubating 50% horse serum, 1.3% sodium dodecyl sulfate, 6 mM Tris-HCl [pH 8.0], 53 SSC, and 0.5 mg of proteinase K per ml for 2 h at 65° C. and then adding 6 mM phenylmethylsulfonyl fluoride, 0.05% sodium azide, and 0.05% Proclin 300) containing 0.70 fmol of preamplifier (described above) per ml, the microwells are cooled and are washed as described above and then incubated at 53° C. for 30 min with 50 μl of preamplifier/amplifier diluent containing 1.0 fmol of bDNA amplifier per ml. After cooling and washing as described above, the microwells are incubated at 53° C. for 15 min with a 50-μl volume of label diluent (preamplifier/amplifier diluent plus 0.85% Brij 35, 0.85 mM ZnC₁₂, and 17 mM MgCl₂) containing 0.40 fmol of label probe per ml. The microwells are cooled for 10 min and then are washed twice with wash A and twice with wash D (0.1 M Tris-HCl [pH 8.0], 2.5 mM MgCl₂, 0.1 mM ZnCl2, 0.1% Brij 35). A 50-μl volume of dioxetane substrate (Lumi-Phos Plus; Lumigen, Detroit, Mich.) is added to each microwell, and after incubation at 37° C. for 30 min, the luminescent output is measured by photon counting in a plate reading luminometer (Chiron Corporation).

The amount of amplification products of the OLA reaction in each specimen is quantified by using a standard curve. The assay standard can consist of a single-stranded DNA molecule. The single-stranded DNA standard is serially diluted in buffer to generate an eight-point standard curve. A calibration curve is generated from a least-squares quadratic polynomial fit in which the dependent variable was the log10 of the signal minus noise and the independent variable was the log10 of the amplification products of the OLA reaction quantification value assignment for each standard. Signal-minus-noise values for both the test samples and standards are calculated by subtracting the geometric mean relative luminescence of two wells containing only Base Matrix from the relative luminescence of each well containing either a sample or a standard.

OLA/PCR product quantification values for each test sample are determined by calculating the mean log10 of the signal-minus-noise value, solving the quadratic equation for the log10 of the OLA/PCR product quantification value, and then inverting back to the arithmetic scale. OLA/PCR product quantification values are expressed in copies, where one copy is defined as the amount of OLA/PCR product in a sample that generates a level of light emission equivalent to that generated by one copy of quality level 1 OLA/PCR product reference material.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a target nucleic acid in a sample comprising
providing a sample potentially containing the target nucleic acid
providing at least one oligonucleotide probe set, wherein said probe set comprises:
(i) a first oligonucleotide probe having a 5′ target specific region and a first 3′ universal sequence region,
(ii) a second oligonucleotide probe having a 3′ target specific region and a second 5′ universal sequence region
(iii) a third oligonucleotide probe having a 5′ region complementary to said first 3′ universal sequence region in said first oligonucleotide probe, and
(v) a fourth oligonucleotide probe having a 3′ region complementary to the 5′ universal sequence region of said second oligonucleotide probe
wherein the first and said second oligonucleotides probes are suitable for ligation together when hybridized adjacent to one another to said target nucleic acid, and wherein the third and the fourth oligonucleotides probes are suitable for ligation to the target nucleic acid when hybridized adjacent to said nucleic acid target;
annealing the oligonucleotide probe set to the target nucleic acid such that a complex is formed between the target nucleic acid and the oligonucleotide probe set;
contacting the complex with a linking agent under conditions such that the directly adjacent 5′ and 3′ ends of the first and second oligonucleotide probes, the 3′ and 5′ ends of the third oligonucleotide probe and the target nucleic acid, and the 5′ and 3′ ends of the fourth oligonucleotide probe and the target nucleic acid covalently bond to form a ligated probe product,
separating the ligated probe product from the non-ligated first and second oligonucleotide probes; and
detecting whether or not said ligated probe product is formed, wherein the presence of the ligated probe product is indicative of presence of said target nucleic acid in said sample.

2. The method of claim 1 wherein the oligonucleotide probes in said oligonucleotide probe set have a predetermined sequence.

3. The method of claim 1 wherein said first oligonucleotide probe comprises in a 3′ to 5′ order said universal region, a tag region and said target specific region.

4. The method of claim 1 wherein said second oligonucleotide probe comprises in a 3′ to 5′ order said target specific region, a tag region and said universal sequence region.

5. The method of claim 3 wherein said third oligonucleotide probe comprises a 3′ region that is complementary to the tag region of said first oligonucleotide probe.

6. The method of claim 3 further comprising a fifth oligonucleotide probe that is complementary to the tag region of said first oligonucleotide probe.

7. The method of claim 4 wherein said fourth oligonucleotide probe comprises a 5′ region that is complementary to the tag region of said second oligonucleotide probe.

8. The method of claim 4 further comprising a fifth oligonucleotide probe that is complementary to the tag region of said second oligonucleotide probe.

9. The method of claim 1 wherein at least one of the universal regions of the first and the second oligonucleotide probe is a promoter sequence.

10. The method of claim 9 wherein the promoter sequence is used as a primer of DNA polymerase.

11. The method of claim 10 wherein said DNA polymerase is selected from the group consisting of Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4DNA polymerase, bacteriophage PR5 DNA polymerase, bacteriophage PR722 DNA polymerase and bacteriophage L17 DNA polymerase.

12. The method of claim 9 wherein the promoter sequence is a promoter for a phage polymerase.

13. The method of claim 12 wherein said phage polymerase is selected from the group consisting of T7 RNA polymerase, T3 RNA polymerase or SP6 RNA polymerase.

14. The method of claim 1 further comprising annealing a first primer complementary to the universal sequence region of the first oligonucleotide probe, contacting the annealed primer with a polymerase under conditions such that the annealed primer is extended to form an extension product complementary to the sequences to which the primers is annealed.

15. The method of claim 14 further comprising detecting the presence of said extension product, wherein the presence of the extended product is indicative of the presence of said target nucleic acid in said sample.

16. The method of claim 14 further comprising annealing a second primer complementary to the universal sequence region of the fourth oligonucleotide probe, contacting the annealed second primer with a polymerase under conditions such that the annealed primer is extended to form an extension product complementary to the sequences to which the primers is annealed.

17. The method of claim 16 further comprising detecting the presence of said extension product, wherein the presence of the extended product is indicative of the presence of the target nucleic acid in said sample.

18. The method of claim 1 further comprising annealing a first primer complementary to the universal sequence region of the fourth oligonucleotide probe, contacting the annealed primer with a polymerase under conditions such that the annealed primer is extended to form extension products complementary to the sequences to which the primers is annealed.

19. The method of claim 18 further comprising detecting the presence of said extension product, wherein the presence of the extended product is indicative of the presence of said target nucleic acid in said sample.

20. The method of claim 15, 16 or 19 wherein said extension product is detected using a DNA microarray, bead microarray, high throughput sequencing or single microtiter plate assay.

21. The method of claim 18 wherein said extension product has a detectable label.

22. The method of claim 21 wherein said detectable label is a fluorescent or biotin label, and the step of detecting includes detecting a fluorescent signal generated by the fluorescent, chemiluminescent or color.

23. The method of claim 21 wherein said label is attached to said primer complementary to said universal sequence region of said first oligonucleotide probe.

24. The method of claim 21 wherein said label is incorporated during the extension of said annealed primer complementary to the universal sequence region of said first oligonucleotide probe.

25. The method of claim 24 wherein said incorporation includes adding a label nucleotide to the extension of the annealed primer complementary to the universal sequence region of said third oligonucleotide probe.

26. The method of claim 1 wherein said universal sequence region of said second oligonucleotide is a phage promoter.

27. The method of claim 26 wherein said phage promoter is selected from the group consisting of T7 RNA polymerase promoter, T3 RNA polymerase promoter or SP6 RNA polymerase promoter.

28. The method of claim 26 further comprising
contacting the phage promoter region of the second oligonucleotide probe with a phage polymerase under conditions such that a transcription product of said phage promoter region is formed
detecting the presence of the transcription product, wherein the presence of the transcription product is indicative of the presence of the target nucleic acid in the sample.

30. The method of claim 28, wherein said transcription product is detected using a DNA microarray, bead microarray, high throughput sequencing or a single microtiter plate assay.

31. The method of claim 28, wherein the transcription product has a detectable label.

32. The method of claim 31 wherein said detectable label is a fluorescent or biotin label, and the step of detecting includes detecting a fluorescent signal generated by the fluorescent or chemiluminescent or color

39. The method of claim 31 wherein said label is incorporated during the transcription of said phage promoter region of said second oligonucleotide probe.

40. The method of claim 39 wherein said incorporation includes adding a label nucleotide to the transcription of said phage promoter region of said second oligonucleotide probe.

41. The method of claim 28 wherein said target nucleic acid is a miRNA molecule.

42. The method of claim 41 wherein said miRNA molecule is derived from total RNA

43. The method of claim 1 wherein said first or third oligonucleotide further comprises a capturing portion.

44. The method of claim 43 wherein said capturing portion is used to separate the ligated probe product from unligated first and second oligonucleotide probes.

45. The method of claim 43 wherein said capturing portion is biotin or a capture sequence.

46. The method of claim 45 wherein said capturing portion is biotin.

47. The method of claim 46 wherein said ligated probe product is isolated by binding said biotin with a strepavidin bound to a solid support.

48. The method of claim 3 or 4 wherein said tag region in said first oligonucleotide probe or said tag in said second oligonucleotide probe are specifically assigned to the target nucleic acid.

49. The method of claim 1 further comprising a loop that links said second and said fourth oligonucleotide.

50. The method of claim 49 further comprising detecting the presence of the ligated probe containing said loop to indicate the presence of said target nucleic acid in said sample.

51. The method claim 506 wherein said detecting comprises binding a branched DNA to said ligated probe.

52. The method of claim 50 wherein said ligated probe is detected using a DNA microarray, bead microarray, or high throughput sequencing.


SEQUENCE LISTING (3 sequences)

SEQ ID NO: 1 - DNA, 22 bp, Artificial Sequence
  Description of Artificial Sequence Synthetic oligonucleotide
tgaggtagtaggttgtatagtt

SEQ ID NO: 2 - DNA, 22 bp, Artificial Sequence
  Description of Artificial Sequence Synthetic oligonucleotide
tgaggtagtaggttgtgtggtt

SEQ ID NO: 3 - DNA, 22 bp, Artificial Sequence
  Description of Artificial Sequence Synthetic oligonucleotide
tgaggtagtaggttgtatggtt